(19) United States
(12) Patent Application Publication
(10) Pub. No.: US 2024/0148554 A1
(43) Pub. Date: May 9, 2024

(54) SYSTEM AND METHODS OF ADJUSTING INTRAOCULAR LENSES WITH OPTICAL COHERENCE TOMOGRAPHY GUIDANCE

(71) Applicant: Alcon Inc., Fribourg (CH)

(72) Inventors: Sumit PALIWAL, Irvine, CA (US); Zsolt BOR, San Clemente, CA (US)

(73) Assignee: Alcon Inc., Fribourg (CH)

(21) Appl. No.: 18/480,896

(22) Filed: Oct. 4, 2023

Related U.S. Application Data
(60) Provisional application No. 63/382,588, filed on Nov. 7, 2022.

(51) Int. Cl. A61F 9/008; G06T 1/00; G06T 7/00

(52) U.S. Cl. CPC A61F 9/00812; G06T 1/0007; G06T 7/0012; A61F 2009/00851; A61F 2009/00887; G06T 2207/10101; G06T 2207/30041

(57) ABSTRACT

Disclosed herein are systems and methods for adjusting an intraocular lens (IOL) with optical coherence tomography (OCT) guidance. The IOL can comprise an optic portion and a plurality of haptics. In one embodiment, a method can comprise directing a laser beam generated by a laser system at a composite material making up part of the IOL. At least part of the composite material can expand in volume in response to the laser beam directed at the composite material. The method can also comprise measuring a volume change of the composite material or a change in at least part of the IOL by analyzing OCT images produced by an OCT imaging apparatus and determining a change in a base power of the IOL based on the measurements.

### CROSS-REFERENCE TO RELATED APPLICATION

This application claims the benefit of U.S. Provisional Application No. 63/382,588 filed on Nov. 7, 2022, the content of which is incorporated herein by reference in its entirety.

### TECHNICAL FIELD

The present disclosure relates generally to the field of intraocular lenses, and, more specifically, to systems and methods for adjusting intraocular lenses (IOLs) with optical coherence tomography (OCT) guidance.

### BACKGROUND

A cataract is a condition involving the clouding over of the normally clear lens of a patient's eye. Cataracts occur as a result of aging, hereditary factors, trauma, inflammation, metabolic disorders, or exposure to radiation. Age-related cataract is the most common type of cataracts. In treating a cataract, the surgeon removes the native crystalline lens matrix from the patient's capsular bag and replaces it with an intraocular lens (IOL). Traditional IOLs provide one or more selected focal lengths that allow the patient to have distance vision. However, after cataract surgery, patients with traditional IOLs often require glasses or other corrective eyewear for certain activities since the eye can no longer undertake accommodation (or change its optical power) to maintain a clear image of an object or focus on an object as its distance varies.

Newer IOLs such as accommodating IOLs, allow the eye to regain at least some focusing ability. Accommodating IOLs (AIOLs) use forces available in the eye to change some portion of the optical system in order to refocus the eye on distant or near targets. Examples of AIOLs are discussed in the following U.S. publications: U.S. Pat. Pub. No. 2021/0100652; U.S. Pat. Pub. No. 2021/0100650; U.S. Pat. Pub. No. 2020/0337833; U.S. Pat. Pub. No. 2018/0256315; U.S. Pat. Pub. No. 2018/0153682; and U.S. Pat. Pub. No. 2017/0049561 and in the following issued U.S. patents: U.S. Pat. Nos. 10,299,913; 10,195,020; and 8,968,396, the contents of which are incorporated herein by reference in their entireties.

There may be a need to adjust AIOLs and non-accommodating IOLs post-operatively or after implantation within the eye of a patient. Therefore, a solution is needed which allows a clinician or other medical professional to safely and accurately adjust an AIOL or non-accommodating IOL post-operatively. Such a solution should be designed with clinical considerations in mind.

### SUMMARY

Disclosed herein are systems and methods for adjusting IOLs with laser light using OCT guidance. In some embodiments, a method of adjusting an IOL with OCT comprises directing a laser beam generated by a laser system at a composite material making up part of the IOL. At least part of the composite material can expand in volume in response to the laser beam directed at the composite material. The method can also comprise measuring a volume change of the composite material by analyzing one or more OCT images of the composite material produced by an OCT imaging apparatus and determining a change in a base power of the IOL based on the volume change of the composite material measured.

In certain embodiments, the step of determining the change in the base power of the IOL further comprises estimating a volume of fluid displaced from either a haptic fluid lumen to an optic fluid chamber or the optic fluid chamber to the haptic fluid lumen in response to the volume change of the composite material measured, and determining the change in the base power of the IOL by selecting a base power change value associated with the volume of fluid displaced from a readout table.

The method can also comprise imaging the IOL using the OCT imaging apparatus and determining a location of the composite material based on the OCT imaging.

In some embodiments, the IOL can comprise at least one haptic including a haptic fluid lumen and a radially-inner haptic lumen wall surrounding at least part of the haptic fluid lumen. The composite material can be configured as a lumen filler making up part of the radially-inner haptic lumen wall and the lumen filler can be configured to expand into at least part of the haptic fluid lumen to reduce a volume of the haptic fluid lumen in response to the laser beam directed at the lumen filler. The composite material can also be configured as a lumen expander making up another part of the radially-inner haptic lumen wall and the lumen expander can be configured to expand to increase the volume of the haptic fluid lumen in response to the laser beam directed at the lumen expander. In these embodiments, the method can further comprise differentiating between the lumen filler and the lumen expander by analyzing the one or more OCT images.

In some embodiments, the method can also comprise adjusting a pulse repetition rate of the laser beam to between about 10 kHz to about 100 kHz. In certain embodiments, the laser beam has a wavelength of between about 1030 nm to about 1064 nm. In some embodiments, the method can further comprise adjusting a laser energy of the laser beam to between about 0.1 μJ to about 100 μJ of laser energy per pulse.

In certain embodiments, the laser beam can be focused by a focusing objective having a numerical aperture of between 0.2 and 0.6. The laser beam can be focused by the focusing objective onto the composite material.

In some embodiments, the method can also comprise controlling the volume change of the composite material by controlling a laser spot diameter created by the laser beam on the composite material. The laser spot diameter can be dictated by the relationship:

$$laser spot diameter = focal point depth * ( 2 * tan ( sin ( \frac{cone angle of laser beam}{2} ) ) ) .$$

The method can further comprise redirecting the laser beam at the composite material using a gonio lens such that the laser beam reaches a part of the IOL obscured by an anatomical structure of the eye.

Another method of adjusting an IOL with OCT is also disclosed. The method can comprise directing a laser beam generated by a laser system at a composite material making up part of the IOL. At least part of the composite material can expand in volume in response to the laser beam directed at the composite material. The IOL can comprise an optic portion including an anterior element and a posterior element. The method can also comprise measuring a change in at least one of a curvature of the anterior element, a curvature of the posterior element, and an axial thickness of the optic portion by analyzing one or more OCT images of the optic portion produced by an OCT imaging apparatus and determining a change in a base power of the IOL based on the measured change in at least one of the curvature of the anterior element, the curvature of the posterior element, and the axial thickness of the optic portion.

In certain embodiments, the step of determining the change in the base power of the IOL further comprises selecting a base power change value associated with the measured change in at least one of the curvature of the anterior element, the curvature of the posterior element, and the axial thickness of the optic portion from a readout table.

In some embodiments, the method can further comprise imaging the IOL including the composite material using the OCT imaging apparatus and determining a location of the composite material based on the OCT imaging.

In some embodiments, the IOL can comprise at least one haptic including a haptic fluid lumen and a radially-inner haptic lumen wall surrounding at least part of the haptic fluid lumen. The composite material can be configured as a lumen filler making up part of the radially-inner haptic lumen wall and the lumen filler can be configured to expand into at least part of the haptic fluid lumen to reduce a volume of the haptic fluid lumen in response to the laser beam directed at the lumen filler. The composite material can also be configured as a lumen expander making up another part of the radially-inner haptic lumen wall and the lumen expander can be configured to expand to increase the volume of the haptic fluid lumen in response to the laser beam directed at the lumen expander. In these embodiments, the method can also comprise differentiating between the lumen filler and the lumen expander by analyzing the one or more OCT images.

In some embodiments, the method can also comprise adjusting a pulse repetition rate of the laser beam to between about 10 kHz to about 100 kHz. In certain embodiments, the laser beam can have a wavelength of between about 1030 nm to about 1064 nm. In some embodiments, the method can further comprise adjusting a laser energy of the laser beam to between about 0.1 μJ to about 100 μJ of laser energy per pulse.

In some embodiments, the method can also comprise controlling the volume change of the composite material by controlling a laser spot diameter created by the laser beam on the composite material. The laser spot diameter can be dictated by the relationship:

$$laser spot diameter = focal point depth * ( 28 tan ( sin ( \frac{cone angle of laser beam}{2} ) ) ) .$$

In certain embodiments, the laser beam can be focused by a focusing objective having a numerical aperture of between 0.2 and 0.6. The laser beam can be focused by the focusing objective onto the composite material.

The method can further comprise redirecting the laser beam at the composite material using a gonio lens such that the laser beam reaches a part of the IOL obscured by an anatomical structure of the eye.

Yet another method of adjusting an IOL with OCT is also disclosed. The method can comprise directing a laser beam generated by a laser system at a composite material making up part of the IOL. At least part of the composite material can expand in volume in response to the laser beam directed at the composite material. The method can also comprise measuring a volume change of a structure or cavity within the IOL in response to the expansion of the composite material by analyzing OCT images of the IOL produced by an OCT imaging apparatus. The method can further comprise determining a change in a base power of the IOL based on the volume change of the structure or cavity measured.

In some embodiments, determining the change in the base power of the IOL can further comprise estimating a volume of fluid displaced from either a haptic fluid lumen to an optic fluid chamber or the optic fluid chamber to the haptic fluid lumen based on a measured volume change of the structure or cavity within the IOL and determining the change in the base power by selecting a base power change value associated with the volume of fluid displaced from a readout table.

Also disclosed is an ophthalmic system for adjusting an IOL post-operatively. The system can comprise a laser system configured to generate a laser beam directed at a composite material making up part of the IOL. At least part of the composite material can expand in volume in response to the laser beam directed at the composite material. The system can also comprise an OCT imaging apparatus configured to produce one or more OCT images of the composite material making up part of the IOL, an image analyzer configured to measure a volume change of the composite material by analyzing the one or more OCT images, and a computing device configured to determine a change in a base power of the IOL based on the volume change of the composite material.

For example, the computing device can estimate a volume of fluid displaced from either a haptic fluid lumen to an optic fluid chamber or the optic fluid chamber to the haptic fluid lumen in response to the volume change of the composite material measured and determine the change in the base power of the IOL by selecting a base power change value associated with the volume of fluid displaced from a readout table.

In some embodiments, the OCT imaging apparatus can be configured to image the IOL prior to the laser beam being directed at the composite material and the computing device can be configured to determine a location of the composite material based on the one or more OCT images of the IOL.

In some embodiments, the IOL can comprise at least one haptic including a haptic fluid lumen and a radially-inner haptic lumen wall surrounding at least part of the haptic fluid lumen. The composite material can be configured as a lumen filler making up part of the radially-inner haptic lumen wall and the lumen filler can be configured to expand into at least part of the haptic fluid lumen to reduce a volume of the haptic fluid lumen in response to the laser beam directed at the lumen filler. The composite material can also be configured as a lumen expander making up another part of the radially-inner haptic lumen wall and the lumen expander can be configured to expand to increase the volume of the haptic fluid lumen in response to the laser beam directed at the lumen expander. In these embodiments, the computing device can be further configured to differentiate between the lumen filler and the lumen expander by analyzing the one or more OCT images.

In some embodiments, the laser beam directed at the composite material can have a pulse repetition rate of between 10 kHz to 100 kHz. In certain embodiments, the laser beam directed at the composite material can have a laser energy per pulse of between about 0.1 μJ to about 100 μJ. In some embodiments, the laser beam can have a wavelength of between about 1030 nm to about 1064 nm. Moreover, in some embodiments, the laser beam can be focused by a focusing objective having a numerical aperture of between 0.2 and 0.6.

In some embodiments, the volume change of the composite material can be controlled in part by a laser spot diameter created by the laser beam on the composite material. The laser spot diameter can be dictated by the relationship:

$$laser spot diameter = focal point depth * ( 2 * tan ( sin ( \frac{cone angle of laser beam}{2} ) ) ) .$$

The ophthalmic system can further comprise a patient interface including a gonio Yen configured to redirect the laser beam at a part of the IOL made up of the composite material that is obscured by an anatomical structure of the eye.

Also disclosed is another ophthalmic system for adjusting an IOL post-operatively. The system can comprise a laser system configured to generate a laser beam directed at a composite material making up part of an intraocular lens (IOL). At least part of the composite material can expand in volume in response to the laser beam directed at the composite material. The IOL can comprise an optic portion including an anterior element and a posterior element. The system can also comprise an optical coherence tomography (OCT) imaging apparatus configured to produce one or more OCT images of the optic portion of the IOL after the laser beam is directed at the composite material and an image analyzer configured to measure a change in at least one of a curvature of the anterior element, a curvature of the posterior element, and an axial thickness of the optic portion by analyzing the one or more OCT images of the optic portion. The system can further comprise a computing device configured to determine a change in a base power of the IOL based on the measured change in at least one of the curvature of the anterior element, the curvature of the posterior element, and the axial thickness of the optic portion.

For example, the computing device can be configured to determine the change in the base power of the IOL by selecting a base power change value associated with the measured change in at least one of the curvature of the anterior element, the curvature of the posterior element, and the axial thickness of the optic portion from a readout table

In some embodiments, the OCT imaging apparatus can be configured to image the IOL prior to the laser beam being directed at the composite material and the computing device can be configured to determine a location of the composite material based on the one or more OCT images of the IOL.

In some embodiments, the IOL can comprise at least one haptic including a haptic fluid lumen and a radially-inner haptic lumen wall surrounding at least part of the haptic fluid lumen. The composite material can be configured as a lumen filler making up part of the radially-inner haptic lumen wall and the lumen filler can be configured to expand into at least part of the haptic fluid lumen to reduce a volume of the haptic fluid lumen in response to the laser beam directed at the lumen filler. The composite material can also be configured as a lumen expander making up another part of the radially-inner haptic lumen wall and the lumen expander can be configured to expand to increase the volume of the haptic fluid lumen in response to the laser beam directed at the lumen expander. In these embodiments, the computing device can be further configured to differentiate between the lumen filler and the lumen expander by analyzing the one or more OCT images.

In some embodiments, the laser beam directed at the composite material can have a pulse repetition rate of between 10 kHz to 100 kHz. In certain embodiments, the laser beam directed at the composite material can have a laser energy per pulse of between about 0.1 μJ to about 100 μJ. In some embodiments, the laser beam can have a wavelength of between about 1030 nm to about 1064 nm. Moreover, in some embodiments, the laser beam can be focused by a focusing objective having a numerical aperture of between 0.2 and 0.6.

In some embodiments, the volume change of the composite material can be controlled in part by a laser spot diameter created by the laser beam on the composite material. The laser spot diameter can be dictated by the relationship:

$$laser spot diameter = focal point depth * ( 2 * tan ( sin ( \frac{cone angle of laser beam}{2} ) ) ) .$$

The ophthalmic system can further comprise a patient interface including a gonio lens configured to redirect the laser beam at a part of the IOL made up of the composite material that is obscured by an anatomical structure of the eye.

Also disclosed is yet another ophthalmic system for adjusting an IOL post-operatively. The system can comprise a laser system configured to generate a laser beam directed at a composite material making up part of the IOL. At least part of the composite material can expand in volume in response to the laser beam directed at the composite material. The system can also comprise an OCT imaging apparatus configured to produce OCT images of the IOL including a structure or cavity within the IOL. The system can also comprise an image analyzer configured to measure a volume change of the structure or cavity within the IOL in response to the expansion of the composite material by analyzing the OCT images. The method can further comprise a computing device configured to determine a change in a base power of the IOL based on a volume change of the structure or cavity measured.

In some embodiments, the computing device can be further configured to estimate a volume of fluid displaced from either a haptic fluid lumen to an optic fluid chamber or the optic fluid chamber to the haptic fluid lumen based on a measured volume change of the structure or cavity within the IOL and determine the change in the base power of the IOL by selecting a base power change value associated with the volume of fluid displaced from a readout table.

### BRIEF DESCRIPTION OF THE DRAWINGS

FIG. 1A illustrate a top plan view of one embodiment of an IOL.

FIGS. 1B and 1C illustrate cross-sectional views of the IOL of FIG. 1A taken along cross-section A-A.

FIG. 1D illustrates an exploded view of the IOL of FIG. 1A.

FIG. 2A illustrates an example of a composite material used to make at least part of the IOL.

FIG. 2B illustrates one embodiment of an expandable component of the composite material increasing in size in response to an external energy applied to the composite material.

FIG. 3 is a block diagram illustrating an OCT-guided laser system configured to adjust the IOL post-operatively.

FIG. 4 illustrates a top-plan view of the IOL with laser spots denoting locations along the lumen fillers where the lumen fillers can be targeted by the laser.

FIGS. 5A and 5B illustrate that a protuberance can be formed at a laser spot where the lumen filler was exposed to the beam of laser light.

FIG. 6A illustrates that the protuberance can form into a shape that resembles a hemisphere.

FIG. 6B illustrates that the protuberance can form into a shape that resembles a paraboloid.

FIG. 6C illustrates that the protuberance can form into a shape that resembles a half-ellipsoid.

FIG. 7A is a cross-sectional OCT image showing a protuberance formed within the haptic.

FIG. 7B is a cross-sectional OCT image showing a segment of a lumen filler with multiple protuberances formed along the segment.

FIG. 7C is a cross-sectional OCT image showing a lumen expander in an expanded configuration.

FIG. 8A is a cross-sectional OCT image showing a portion of a haptic of the IOL with a lumen filler and a lumen expander visible in the OCT image.

FIG. 8B is an en face OCT image showing a top-down view of a portion of the haptic with the composite material visible in the OCT image.

FIGS. 9A and 9B are OCT images showing a change in an axial thickness of the optic portion in response to fluid entering the optic fluid chamber.

FIG. 9C illustrates that at least one of the anterior element and the posterior element can change its curvature in response to fluid entering or exiting the optic fluid chamber.

FIG. 10 illustrates a gonio lens being used to re-direct laser light to more precisely target the composite material without harming the iris of the eye.

### DETAILED DESCRIPTION

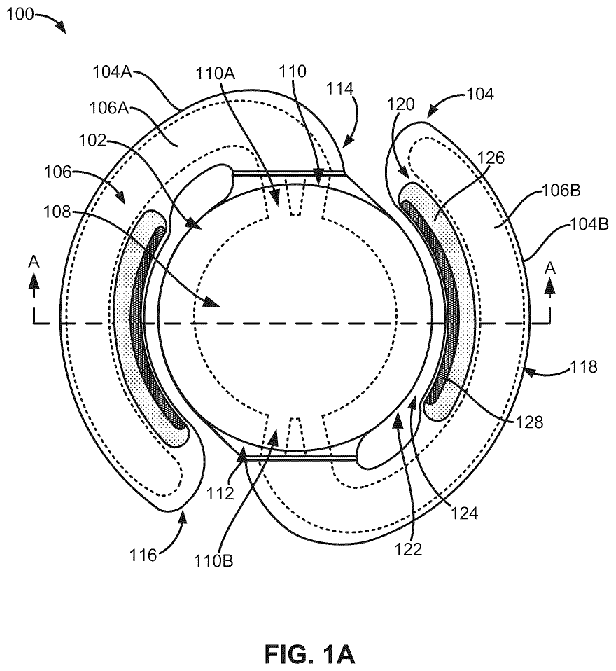

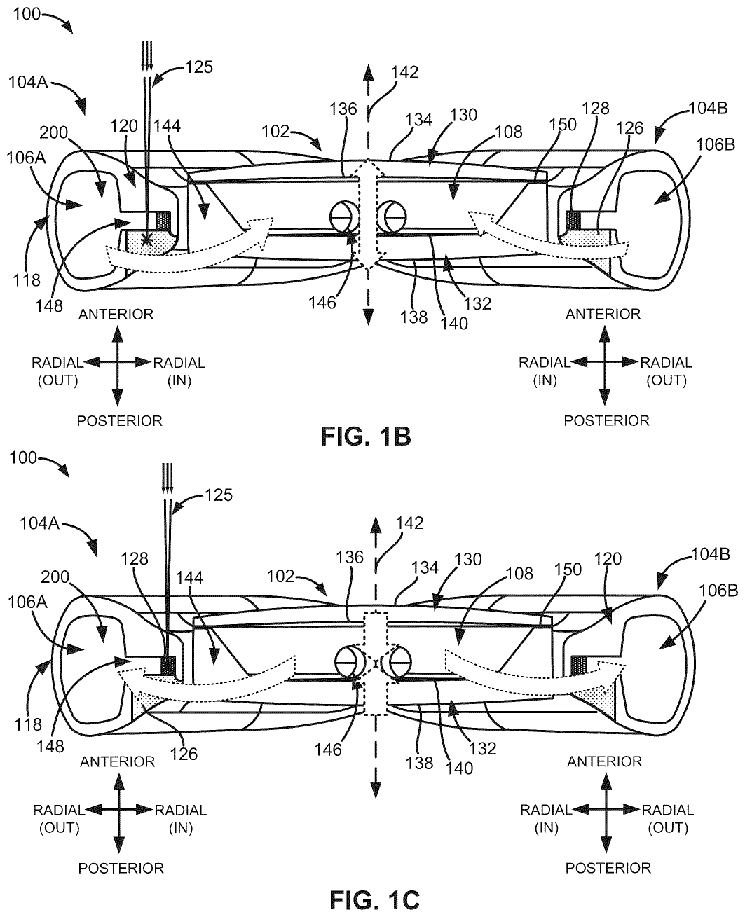

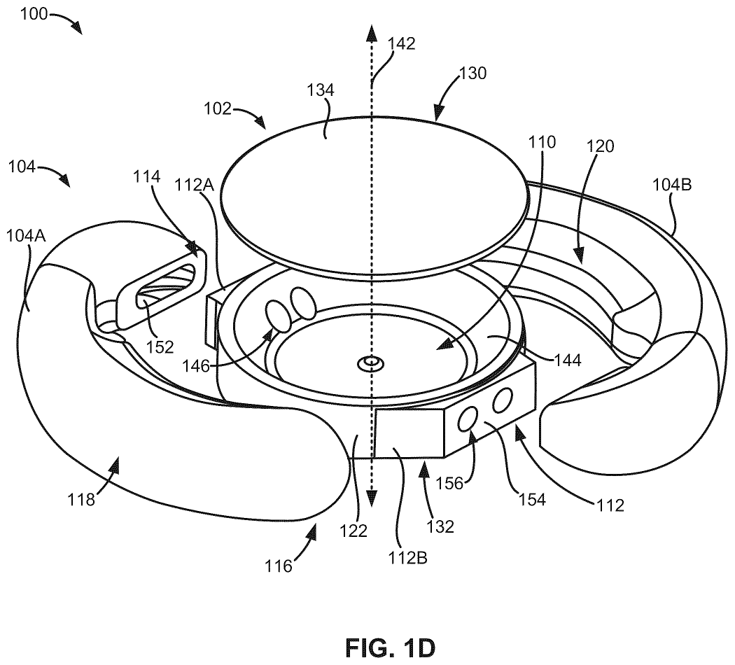

FIG. 1A illustrates a top plan view of one embodiment of an adjustable intraocular lens (IOL) 100. For example, the adjustable IOL can be an adjustable accommodating IOL (AIOL). The IOL 100 can be implanted within a subject to correct for defocus aberration, corneal astigmatism, spherical aberration, or a combination thereof. The IOL 100 can comprise an optic portion 102 and one or more haptics 104 including a first haptic 104A and a second haptic 104B coupled to and extending peripherally from the optic portion 102. The IOL 100 can be positioned within a native capsular bag in which a native lens has been removed.

In some embodiments, the haptics 104 can be coupled to and adhered to the optic portion 102. For example, the haptics 104 can be adhered to the optic portion 102 after each is formed separately. In other embodiments, the IOL 100 can be a one-piece lens such that the haptics 104 are connected to and extend from the optic portion 102. In this example embodiment, the haptics 104 are formed along with the optic portion 102 and are not adhered or otherwise coupled to the optic portion 102 in a subsequent step.

The IOL 100 can be implanted within a native capsular bag of a subject after the subject's native lens has been removed. When implanted within the native capsular bag, the optic portion 102 can be adapted to refract light that enters the eye onto the retina. The one or more haptics 104 can be configured to engage the capsular bag and be adapted to deform in response to ciliary muscle movement (e.g., muscle relaxation, muscle contraction, or a combination thereof) in connection with capsular bag reshaping.

Each of the haptics 104 can comprise a haptic fluid lumen 106 extending through at least part of the haptic 104. For example, the first haptic 104A can comprise a first haptic fluid lumen 106A extending through at least part of the first haptic 104A and the second haptic 104B can comprise a second haptic fluid lumen 106B extending through at least part of the second haptic 104B. The haptic fluid lumen 106 (e.g., any of the first haptic fluid lumen 106A or the second haptic fluid lumen 106B) can be in fluid communication with or fluidly connected to an optic fluid chamber 108 within the optic portion 102.

The optic fluid chamber 108 can be in fluid communication with the one or more haptic fluid lumens 106 through one or more fluid channels 110. The fluid channels 110 can be conduits or passageways fluidly connecting the optic fluid chamber 108 to the haptic fluid lumens 106. The fluid channels 110 can be spaced apart from one another. For example, a pair of fluid channels 110 can be spaced apart between about 0.1 mm to about 1.0 mm. In some embodiments, each of the fluid channels 110 can have a diameter of between about 0.4 mm to about 0.6 mm.

The haptics 104 can be coupled to the optic portion 102 at a reinforced portion 112. The reinforced portion 112 can serve as a haptic-optic interface. The pair of fluid channels 110 can be defined or formed within part of the reinforced portion 112.

As shown in FIG. 1A, the optic fluid chamber 108 can be in fluid communication with the first haptic fluid lumen 106A through a first pair of fluid channels 110A. The optic fluid chamber 108 can also be in fluid communication with the second haptic fluid lumen 106B through a second pair of fluid channels 110B.

In some embodiments, the first pair of fluid channels 110A and the second pair of fluid channels 110B can be positioned substantially on opposite sides of the optic portion 102. The first pair of fluid channels 110A can be positioned substantially diametrically opposed to the second pair of fluid channels 110B. The first pair of fluid channels 110A and the second pair of fluid channels 110B can be defined or extend through part of the optic portion 102. The first pair of fluid channels 110A and the second pair of fluid channels 110B can be defined or extend through a posterior element 132 of the optic portion 102 (see, e.g., FIGS. 1B-1D).

FIG. 1A also illustrates that each of the haptics 104 (e.g., any of the first haptic 104A or the second haptic 104B) can have a proximal attachment end 114 and a distal free end 116. A haptic fluid port 152 (see, e.g., FIG. 1C) can be defined at the proximal attachment end 114 of the haptic 104. The haptic fluid port 152 can serve as an opening of the haptic fluid lumen 106. Fluid within the haptic fluid lumen 106 can flow out of the haptic fluid lumen 106 through the haptic fluid port 152 and into the optic fluid chamber 108 via the fluid channels 110 when the haptic 104 is coupled to the optic portion 102. Similarly, fluid within the optic fluid chamber 108 can flow out of the optic fluid chamber 108 through the pair of fluid channels 110 and into the haptic fluid lumen 106 through the haptic fluid port 152.

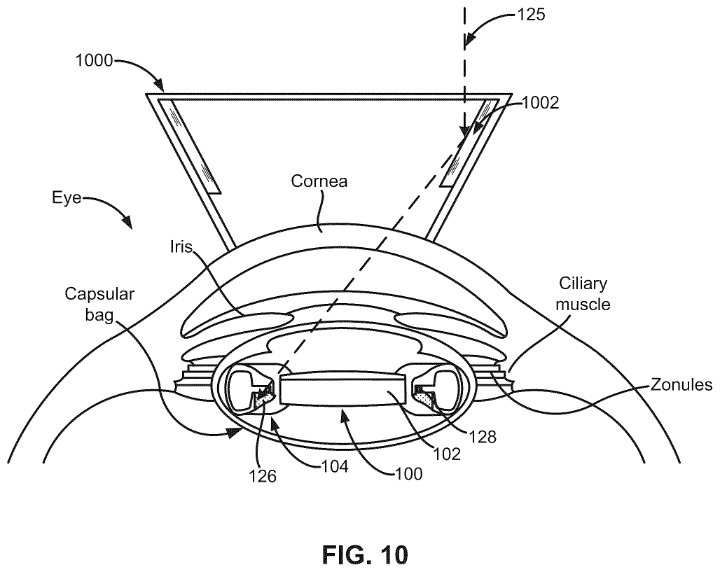

Each of the haptics 104 can comprise a radially-outer haptic lumen wall 118 and a radially-inner haptic lumen wall 120. The radially-outer haptic lumen wall 118 (also referred to as a radially-outer lateral wall of the haptic 104) can be configured to face and contact an inner surface of a patient's capsular bag (see, e.g., FIG. 10) when the IOL 100 is implanted within the capsular bag. The radially-inner haptic lumen wall 120 (also referred to as a radially-inner lateral wall of the haptic 104) can be configured to face an outer peripheral surface 122 of the optic portion 102.

As previously discussed, the IOL 100 can be implanted or introduced into a patient's capsular bag after a native lens has been removed from the capsular bag. The patient's capsular bag is connected to zonule fibers which are connected to the patient's ciliary muscles (see, e.g., FIG. 10). The capsular bag is elastic and ciliary muscle movements can reshape the capsular bag via the zonule fibers. For example, when the ciliary muscles relax, the zonules are stretched. This stretching pulls the capsular bag in the generally radially outward direction due to radially outward forces. This pulling of the capsular bag causes the capsular bag to elongate, creating room within the capsular bag. When the patient's native lens is present in the capsular bag, the native lens normally becomes flatter (in the anterior-to-posterior direction), which reduces the power of the lens, allowing for distance vision. In this configuration, the patient's native lens is said to be in a disaccommodated state or undergoing disaccommodation.

When the ciliary muscles contract, however, as occurs when the eye is attempting to focus on near objects, the radially inner portion of the muscles move radially inward, causing the zonules to slacken. The slack in the zonules allows the elastic capsular bag to contract and exert radially inward forces on a lens within the capsular bag. When the patient's native lens is present in the capsular bag, the native lens normally becomes more curved (e.g., the anterior part of the lens becomes more curved), which gives the lens more power, allowing the eye to focus on near objects. In this configuration, the patient's native lens is said to be in an accommodated state or undergoing accommodation.

In embodiments where the IOL 100 is an AIOL, the radially-outer haptic lumen wall 118 of the implanted AIOL can directly engage with or be in physical contact with the portion of the capsular bag that is connected to the zonules or zonule fibers. Therefore, the radially-outer haptic lumen wall 118 of the AIOL can be configured to respond to capsular bag reshaping forces that are applied radially when the zonules relax and stretch as a result of ciliary muscle movements.

For example, when the ciliary muscles contract, the peripheral region of the elastic capsular bag reshapes and applies radially inward forces on the radially-outer haptic lumen wall 118 of each of the haptics 104. When the IOL 100 is an AIOL, the radially-outer haptic lumen wall 118 can deform or otherwise change shape and this deformation or shape-change can cause the volume of the haptic fluid lumen 106 to decrease. When the volume of the haptic fluid lumen 106 decreases, the fluid within the haptic fluid lumen 106 is moved or pushed into the optic fluid chamber 108. The optic portion 102 of the AIOL can change shape in response to fluid entering the optic fluid chamber 108 from the haptic fluid lumen 106. This can increase the base power or base spherical power of the AIOL and allow a patient with the AIOL implanted within the eye of the patient to focus on near objects. In this state, the adjustable AIOL can be considered to have undergone accommodation.

When the ciliary muscles relax, the peripheral region of the elastic capsular bag is stretched radially outward and the capsular bag elongates and more room is created within the capsular bag. The radially-outer haptic lumen wall 118 of the haptics 104 can be configured to respond to this capsular bag reshaping by returning to its non-deformed or non-stressed configuration. This causes the volume of the haptic fluid lumen 106 to increase or return to its non-deformed volume. This increase in the volume of the haptic fluid lumen 106 can cause the fluid within the optic fluid chamber 108 to be drawn out or otherwise flow out of the optic fluid chamber 108 and back into the haptic fluid lumen 106. Fluid moves out of the optic fluid chamber 108 into the haptic fluid lumen 106 through the same fluid channels 110 formed within the optic portion 102.

The optic portion 102 of the AIOL can change shape in response to fluid exiting the optic fluid chamber 108 and into the haptic fluid lumen 106. This can decrease the base power or base spherical power of the AIOL and allow a patient with the AIOL implanted within the eye of the patient to focus on distant objects or provide for distance vision. In this state, the AIOL can be considered to have undergone disaccommodation.

When the IOL 100 is an AIOL, the radially-outer haptic lumen walls 118 of the haptics 104 can be made thinner than the radially-inner haptic lumen walls 120 to allow the haptics to maintain a high degree of sensitivity to radial forces applied to an equatorial region of the haptics 104 by capsular bag reshaping as a result of ciliary muscle movements. As shown in FIGS. 1B and 1C, the radially-inner haptic lumen walls 120 of the haptics 104 can be designed to be thicker or bulkier than the radially-outer haptic lumen walls 118 to provide the haptics 104 with stiffness or resiliency in the anterior-to-posterior direction. In certain embodiments, the radially-inner haptic lumen wall 120 can taper in shape as the radially-inner haptic lumen wall 120 gets closer to the optic portion 102. When designed in this manner, the haptics 104 can be less sensitive to capsular bag forces applied in the anterior-to-posterior direction. For example, when capsular bag forces are applied to the haptics 104 in the anterior-to-posterior direction, less fluid movement occurs between the haptic fluid lumens 106 and the optic fluid chamber 108 than when forces are applied in the radial direction. Since less fluid movement occurs, less changes in the base power of the AIOL occur.

Although FIGS. 1A-1D illustrate the IOL 100 as an AIOL, it is contemplated by this disclosure that the IOL 100 can also be a non-accommodating or static-focus adjustable IOL and that the OCT-guided laser system 300 can also be used to adjust the base power of the non-accommodating or static-focus adjustable IOL. Examples of non-accommodating or static-focus adjustable IOLs are discussed in U.S. Pat. Pub. No. 2021/0100649, the content of which is incorporated herein by reference in its entirety.

In some embodiments, the IOL 100 can be designed such that a gap 124 or void space radially separates the radially-inner haptic lumen wall 120 of the haptic 104 from the outer peripheral surface 122 of the optic portion 102. This can allow portions of the haptic 104 to change shape or expand in response to an external energy such as a laser light 125 (see, e.g., FIGS. 1B and 1C) directed at the haptic 104.

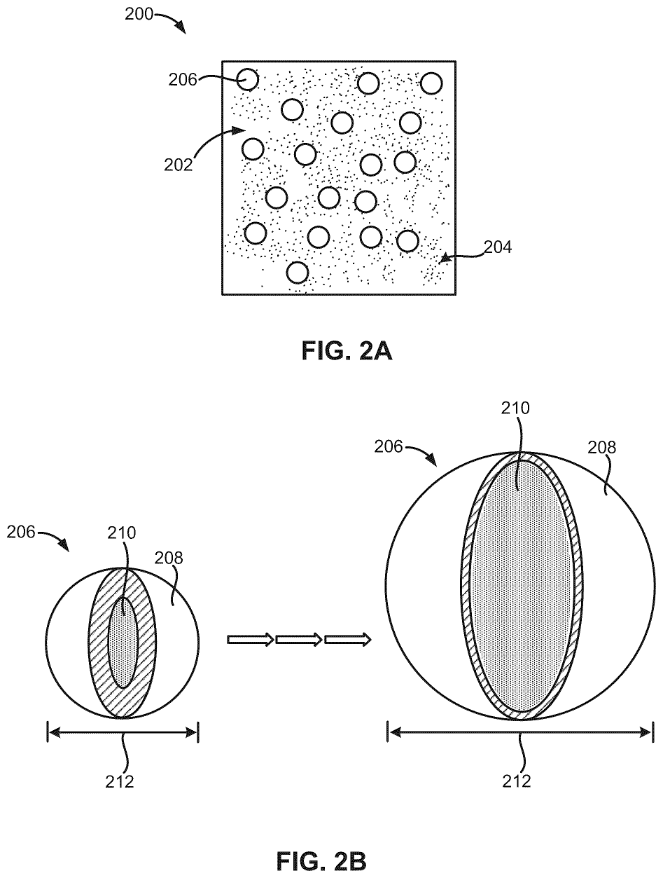

FIG. 1A also illustrates that one or more portions of each of the haptics 104 can be made of a composite material 200 (see, e.g., FIG. 2A). As will be discussed in more detail in later sections, the composite material 200 can comprise or be made in part of an energy absorbing constituent, a plurality of expandable components, and a cross-linked copolymer used to make the rest of the haptic 104. The portions of the haptics 104 made of the composite material 200 can be configured to expand in response to the laser light 125 (see, e.g., FIGS. 1B and 1C) directed at the composite material 200. Depending on where the composite material 200 is positioned or integrated within each of the haptics 104, the composite material 200 can act as a lumen filler 126 to take up space within the haptic fluid lumen 106 or a lumen expander 128 to create more space within the haptic fluid lumen 106.

As will be discussed in more detail in later sections, when laser light 125 is applied to the composite material 200 configured as the lumen filler 126, the composite material 200 can expand and the expansion of the composite material 200 in this instance can decrease a volume of the haptic fluid lumen 106 and cause fluid within the haptic fluid lumen 106 to be displaced into the optic fluid chamber 108. This can cause the optic portion 102 to change shape (e.g., cause the anterior or posterior elements of the optic portion 102 to become more curved) leading to an increase in the base power of the optic portion 102.

Alternatively, when the laser light 125 is applied to the composite material 200 configured as the lumen expander 128, the composite material 200 can expand and the expansion of the composite material 200 in this instance can increase a volume of the haptic fluid lumen 106 and cause fluid within the optic fluid chamber 108 to be drawn into the haptic fluid lumen 106. This can also cause the optic portion 102 to change shape (e.g., cause the anterior or posterior elements of the optic portion 102 to become less curved or flatter) leading to a decrease in the base power of the optic portion 102.

FIGS. 1B and 1C illustrate cross-sectional views of the IOL 100 of FIG. 1A taken along cross-section A-A. As shown in FIGS. 1B and 1C, the optic portion 102 can comprise an anterior element 130 and a posterior element 132. The fluid-filled optic fluid chamber 108 can be defined in between the anterior element 130 and the posterior element 132.

The anterior element 130 can comprise an anterior optical surface 134 and an anterior inner surface 136 opposite the anterior optical surface 134. The posterior element 132 can comprise a posterior optical surface 138 and a posterior inner surface 140 opposite the posterior optical surface 138. Any of the anterior optical surface 134, the posterior optical surface 138, or a combination thereof can be considered and referred to as an external optical surface. The anterior inner surface 136 and the posterior inner surface 140 can face the optic fluid chamber 108. At least part of the anterior inner surface 136 and at least part of the posterior inner surface 140 can serve as chamber walls of the optic fluid chamber 108.

As shown in FIGS. 1B and 1C, the optic portion 102 can have an optical axis 142 extending in an anterior-to-posterior direction through a center of the optic portion 102. The optical axis 142 can extend through the centers of both the anterior element 130 and the posterior element 132.

The thickness of the anterior element 130 can be greater at or near the optical axis 142 than at the periphery of the anterior element 130. In some embodiments, the thickness of the anterior element 130 can increase gradually from the periphery of the anterior element 130 toward the optical axis 142.

In certain embodiments, the thickness of the anterior element 130 at or near the optical axis 142 can be between about 0.45 mm and about 0.55 mm. In these and other embodiments, the thickness of the anterior element 130 near the periphery can be between about 0.20 mm and about 0.40 mm. Moreover, the anterior inner surface 136 of the anterior element 130 can have less curvature or be flatter than the anterior optical surface 134.

The thickness of the posterior element 132 can be greater at or near the optical axis 142 than portions of the posterior element 132 radially outward from the optical axis 142 but prior to reaching a raised periphery 144 of the posterior element 132. The thickness of the posterior element 132 can gradually decrease from the optical axis 142 to portions radially outward from the optical axis 142 (but prior to reaching the raised periphery 144). As shown in FIGS. 1B and 1C, the thickness of the posterior element 132 can increase once again from a radially inner portion of the raised periphery 144 to a radially outer portion of the raised periphery 144.

In certain embodiments, the thickness of the posterior element 132 at or near the optical axis 142 can be between about 0.45 mm and about 0.55 mm. In these and other embodiments, the thickness of the posterior element 132 radially outward from the optical axis 142 (but prior to reaching the raised periphery 144) can be between about 0.20 mm and about 0.40 mm. The thickness of the posterior element 132 near the radially outer portion of the raised periphery 144 can be between about 1.00 mm and 1.15 mm. Moreover, the posterior inner surface 140 of the posterior element 132 can have less curvature or be flatter than the posterior optical surface 138.

The optic portion 102 can have a base power or base spherical power. The base power of the optic portion 102 can be configured to change based on an internal fluid pressure within the fluid-filled optic fluid chamber 108. The base power of the optic portion 102 can be configured to increase or decrease as fluid enters or exits the fluid-filled optic fluid chamber 108.

The base power of the optic portion 102 can be configured to increase as fluid enters the fluid-filled optic fluid chamber 108 from the haptic fluid lumen(s) 106, as depicted in FIG. 1B using the curved broken-line arrows. For example, the anterior element 130 of the optic portion 102 can be configured to increase its curvature in response to the fluid entering the optic fluid chamber 108. Also, for example, the posterior element 132 of the optic portion 102 can be configured to increase its curvature in response to the fluid entering the optic fluid chamber 108. In further embodiments, both the anterior element 130 and the posterior element 132 can be configured to increase their curvatures in response to the fluid entering the optic fluid chamber 108.

The base power of the optic portion 102 can be configured to decrease as fluid exits or is drawn out of the fluid-filled optic fluid chamber 108 into the haptic fluid lumen(s) 106, as depicted in FIG. 1C using the curved broken-line arrows. For example, the anterior element 130 of the optic portion 102 can be configured to decrease its curvature (or flatten out) in response to the fluid exiting the optic fluid chamber 108. Also, for example, the posterior element 132 of the optic portion 102 can be configured to decrease its curvature (or flatten out) in response to the fluid exiting the optic fluid chamber 108. In further embodiments, both the anterior element 130 and the posterior element 132 can be configured to decrease their curvatures in response to the fluid exiting the optic fluid chamber 108.

It should be noted that although FIGS. 1B and 1C illustrate the fluid entering and exiting the optic fluid chamber 108 from the haptic fluid lumens 106 using the curved broken-line arrows, fluid enters and exits the optic fluid chamber 108 via the fluid channels 110 and apertures 146 defined along the posterior element 132. The apertures 146 can be holes or openings defined along the posterior element 132 that serve as terminal ends of the fluid channels 110. When the IOL 100 comprises a pair of fluid channels 110, the pair of apertures serving as ends of the fluid channels 110 can be spaced apart from one another between about 0.1 mm to about 1.0 mm.

As shown in FIGS. 1B and 1C, one or more portions of the IOL 100 can be made of a composite material 200 (see, e.g., FIG. 2A) designed to respond to an external energy, such as laser light 125, applied to the composite material 200. For example, one or more portions of each of the haptics 104 of the IOL 100 can be made of the composite material 200.

Depending on where the composite material 200 is positioned or integrated within each of the haptics 104, the composite material 200 can act as a lumen filler 126 or a lumen expander 128. As shown in FIGS. 1A-1C, the same IOL 100 can comprise both lumen fillers 126 and lumen expanders 128.

The lumen filler 126 can be a portion of the haptic 104 made of the composite material that is designed to decrease a volume of the haptic fluid lumen 106 in response to an external energy (e.g., laser light 125) directed at the lumen filler 126. The lumen expander 128 can be a portion of the haptic 104 made of the composite material 200 that is designed to increase a volume of the haptic fluid lumen 106 in response to an external energy (e.g., laser light 125) directed at the lumen expander 128.

As shown in FIGS. 1B and 1C, each of the haptics 104 can comprise a channel 148. The channel 148 can be defined within part of the radially-inner haptic lumen wall 120. For example, the channel 148 can extend partially into the radially-inner haptic lumen wall 120. The channel 148 can be in fluid communication with the haptic fluid lumen 106 or be considered part of the haptic fluid lumen 106.

In some embodiments, the lumen filler 126 can be positioned posterior to the channel 148. In these embodiments, the lumen filler 126 can replace or act as the posterior portion of the radially-inner haptic lumen wall 120. The lumen filler 126 can also be positioned radially inward of the portion of the haptic fluid lumen 106 that is not the channel 148.

At least part of the lumen filler 126 can be in fluid communication with the channel 148. For example, at least part of an anterior portion or layer of the lumen filler 126 can be in fluid communication with or otherwise exposed to the channel 148.

As shown in FIGS. 1B and 1C, in some embodiments, a radially outer lateral side of the lumen filler 126 is not in fluid communication with the haptic fluid lumen 106. In these embodiments, the radially outer lateral side of the lumen filler 126 is separated from the haptic fluid lumen 106 by a part of the haptic 104 not made of the composite material 200.

The lumen expander 128 can be positioned radially inward of the channel 148. The lumen expander 128 can also be positioned anterior to the lumen filler 126. More specifically, for example, the lumen expander 128 can be positioned anterior to a radially inner portion of the lumen filler 126.

In some embodiments, the lumen expander 128 can be positioned within the channel 148. In these embodiments, the lumen expander 128 can be positioned at a radially innermost end of the channel 148. For example, the radially-inner haptic lumen wall 120 can taper in shape as the radially-inner haptic lumen wall 120 gets closer to the optic portion 102. The lumen expander 128 can be positioned at a radially innermost end of the channel 148 near the tapered end of the radially-inner haptic lumen wall 120.

As shown in FIGS. 1B and 1C, a radially outer lateral side of the lumen expander 128 can be in fluid communication with the channel 148 and the haptic fluid lumen 106. In some embodiments, the lumen expander 128 does not extend all the way to the radially inner-most part of the radially-inner haptic lumen wall 120. In these embodiments, a part of the haptic 104 that is not made of the composite material 200 can serve as the radially inner-most part of the radially-inner haptic lumen wall 120 and separate the lumen expander 128 from the outer peripheral surface 122 of the optic portion 102.

In some embodiments, the lumen expander 128 can be connected or otherwise coupled to the lumen filler 126. In these and other embodiments, the lumen expander 128 and the lumen filler 126 can be or refer to different parts of the same composite material 200. For example, the lumen filler 126 can be shaped substantially as a curved cornice and the lumen expander 128 can be shaped substantially as a rectangular cuboid extending from an anterior surface of the cornice.

It should be understood by one of ordinary skill in the art that even though different colored shading is used to differentiate the lumen filler 126 from the lumen expander 128 in the figures (that is, a darker shading pattern is used to depict the lumen expander 128 and a lighter shading pattern is used to depict the lumen filler 126), both the lumen filler 126 and the lumen expander 128 can be made of the same composite material 200 or refer to different parts/features of the same block of composite material 200.

In other embodiments, the lumen filler 126 and the lumen expander 128 can be made of different types of composite materials 200. In these embodiments, the lumen filler 126 can be made of a first type of composite material 200 and the lumen expander 128 can be made of a second type of composite material 200. In certain embodiments, the lumen filler 126 and the lumen expander 128 can be made of different colored composite materials 200. For example, the composite material 200 can comprise an energy absorbing constituent such as an energy absorbing pigment or dye.

As a more specific example, either the lumen filler 126 or the lumen expander 128 can be made of a composite material 200 comprising a black-colored energy absorbing pigment such as graphitized carbon black. In this example, if one of the lumen filler 126 or the lumen expander 128 is made of a composite material 200 comprising graphitized carbon black, the other can be made of another type of composite material 200 comprising a red-colored energy absorbing pigment such as an azo dye (e.g., Disperse Red 1 dye).

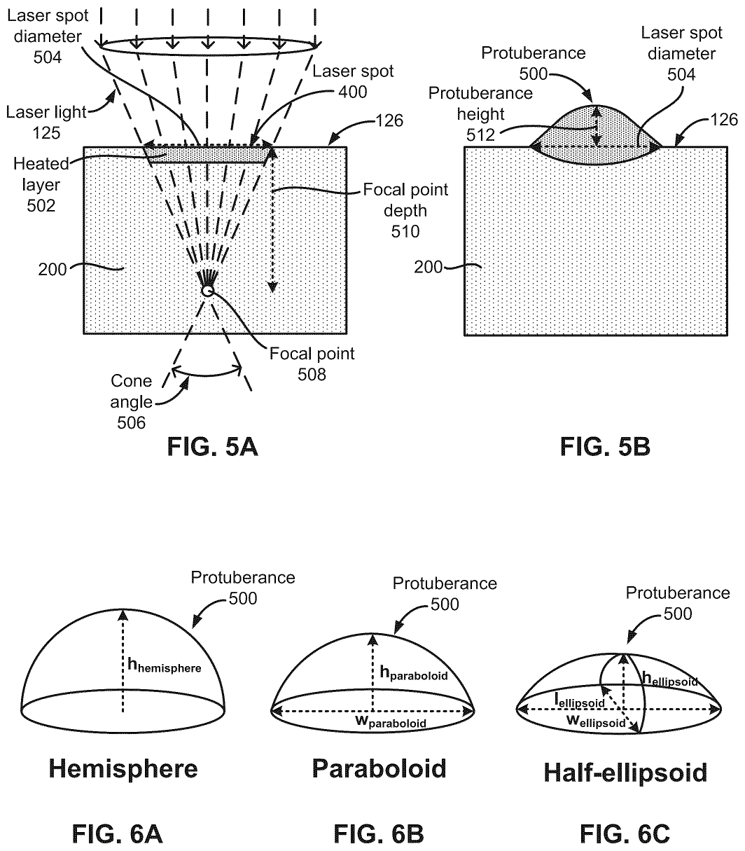

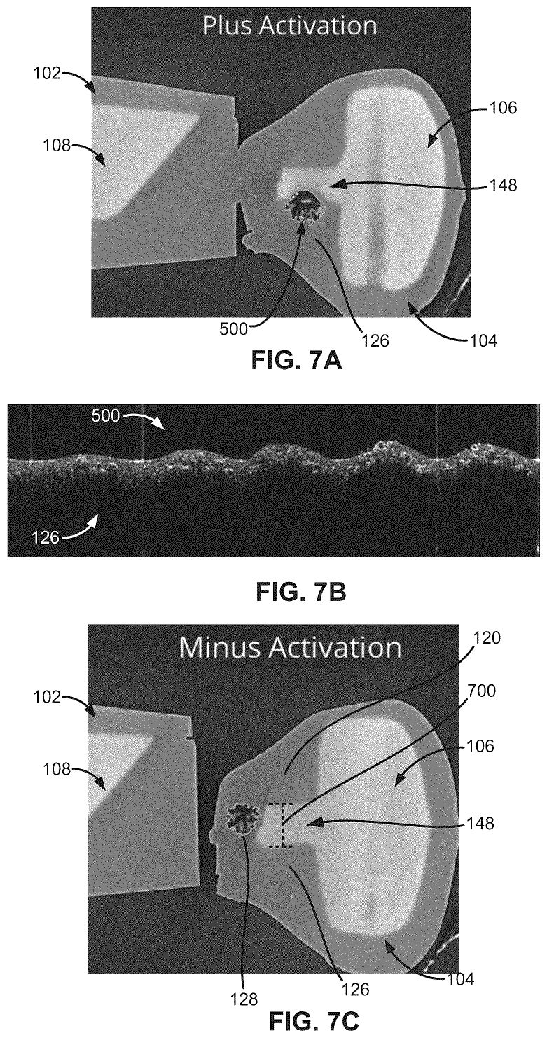

As shown in FIG. 1B, an external energy such as laser light 125 can be directed at the lumen filler 126 to cause at least part of the lumen filler 126 to expand and grow in size. As will be discussed in more detail in later sections, this expansion can manifest itself as a protuberance 500 (see, e.g., FIGS. 5B, 7A, and 7B) growing or jutting out of the lumen filler 126. For example, when laser light 125 is directed at the anterior portion or layer of the lumen filler 126 in fluid communication with or otherwise exposed to the channel 148, a protuberance 500 can grow out of the anterior portion and into the channel 148. Since the channel 148 is in fluid communication with the haptic fluid lumen 106 (or is considered part of the haptic fluid lumen 106), the volume of the haptic fluid lumen 106 can decrease in response to the formation of the protuberance 500. This can cause fluid within the haptic fluid lumen 106 to be pushed or otherwise displaced into the optic fluid chamber 108. As a result, at least one of the anterior element 130 and the posterior element 132 can increase its curvature and the base power of the optic portion 102 can increase in response to the laser light 125 directed at the lumen filler 126.

An external energy such as the laser light 125 can be directed at the lumen expander 128 to cause at least part of the lumen expander 128 to expand and grow in size. As will be discussed in more detail in later sections, this expansion can manifest itself as an expansion of the channel 148 (see, e.g., FIG. 7C). For example, when laser light 125 is directed at the lumen expander 128, the lumen expander 128 can grow in size and enlarge the channel 148. Since the channel 148 is in fluid communication with the haptic fluid lumen 106 (or is considered part of the haptic fluid lumen 106), the volume of the haptic fluid lumen 106 can increase in response to the growth of the lumen expander 128. This can cause fluid within the haptic fluid lumen 106 to be drawn out of the optic fluid chamber 108 and into the haptic fluid lumen 106. As a result, at least one of the anterior element 130 and the posterior element 132 can decrease its curvature and the base power of the optic portion 102 can decrease in response to the laser light 125 directed at the lumen expander 128.

One technical problem faced by the applicants is that once an IOL 100 is implanted within a capsular bag of a patient, an aggressive healing response by tissue within the capsular bag can squeeze or contract the optic portion 102 of the lens and drive the optical power higher than initially anticipated. Another technical problem faced by the applicants is that the pre-operative biometry measurements made on a patient's eye may be incorrect, leading to lenses with the wrong lens power being prescribed and implanted within the patient. Moreover, yet another technical problem faced by the applicants is that a patient's cornea or muscles within the eye may change as a result of injury, disease, or aging. One technical solution discovered and developed by the applicants is to design an IOL 100 that can be adjusted post-operatively (i.e., post-implantation) to account for such changes or errors.

In some embodiments, the fluid within the optic fluid chamber 108 and the haptic fluid lumen(s) 106 can be an oil. More specifically, in certain embodiments, the fluid within the optic fluid chamber 108 and the haptic fluid lumen(s) 106 can be a silicone oil or fluid. For example, the fluid can be a silicone oil made in part of a diphenyl siloxane. In other embodiments, the fluid can be a silicone oil made in part of a ratio of two dimethyl siloxane units to one diphenyl siloxane unit. More specifically, in some embodiments, the fluid can be a silicone oil made in part of diphenyltetramethyl cyclotrisiloxane or a copolymer of diphenyl siloxane and dimethyl siloxane. In further embodiments, the fluid can be a silicone oil comprising branched polymers.

The fluid (e.g., the silicone oil) can be index matched with a lens body material used to make the optic portion 102. When the fluid is index matched with the lens body material, the entire optic portion 102 containing the fluid can act as a single lens. For example, the fluid can be selected so that it has a refractive index of between about 1.48 and 1.53 (or between about 1.50 and 1.53). In some embodiments, the fluid (e.g., the silicone oil) can have a polydispersity index of between about 1.2 and 1.3. In other embodiments, the fluid (e.g., the silicone oil) can have a polydispersity index of between about 1.3 and 1.5. In other embodiments, the fluid (e.g., the silicone oil) can have a polydispersity index of between about 1.1 and 1.2. Other example fluids are described in U.S. Patent Publication No. 2018/0153682, which is herein incorporated by reference in its entirety.

The optic portion 102 can be made in part of a deformable or flexible material. In some embodiments, the optic portion 102 can be made in part of a deformable or flexible polymeric material. For example, the anterior element 130, the posterior element 132, or a combination thereof can be made in part of a deformable or flexible polymeric material. The one or more haptics 104 (e.g., the first haptic 104A, the second haptic 104B, or a combination thereof) can be made in part of the same deformable or flexible material as the optic portion 102. In other embodiments, the one or more haptics 104 can be made in part of different materials from the optic portion 102.

In some embodiments, the optic portion 102 can comprise or be made in part of a lens body material. The lens body material can be made in part of a cross-linked copolymer comprising a copolymer blend. The copolymer blend can comprise an alkyl acrylate or methacrylate, a fluoro-alkyl (meth)acrylate, and a phenyl-alkyl acrylate. It is contemplated by this disclosure and it should be understood by one of ordinary skill in the art that these types of acrylic cross-linked copolymers can be generally copolymers of a plurality of acrylates, methacrylates, or a combination thereof and the term “acrylate” as used herein can be understood to mean acrylates, methacrylates, or a combination thereof interchangeably unless otherwise specified. The cross-linked copolymer used to make the lens body material can comprise an alkyl acrylate in the amount of about 3% to 20% (wt %), a fluoro-alkyl acrylate in the amount of about 10% to 35% (wt %), and a phenyl-alkyl acrylate in the amount of about 50% to 80% (wt %). In some embodiments, the cross-linked copolymer can comprise or be made in part of an n-butyl acrylate as the alkyl acrylate, trifluoroethyl methacrylate as the fluoro-alkyl acrylate, and phenylethyl acrylate as the phenyl-alkyl acrylate. More specifically, the cross-linked copolymer used to make the lens body material can comprise n-butyl acrylate in the amount of about 3% to 20% (wt %) (e.g., between about 12% to 16%), trifluoroethyl methacrylate in the amount of about 10% to 35% (wt %) (e.g., between about 17% to 21%), and phenylethyl acrylate in the amount of about 50% to 80% (wt %) (e.g., between about 64% to 67%).

The final composition of the cross-linked copolymer used to make the lens body material can also comprise a cross-linker or cross-linking agent such as ethylene glycol dimethacrylate (EGDMA). For example, the final composition of the cross-linked copolymer used to make the lens body material can also comprise a cross-linker or cross-linking agent (e.g., EGDMA) in the amount of about 1.0%. The final composition of the cross-linked copolymer used to make the lens body material can also comprise an initiator or initiating agent (e.g., Perkadox 16) and a UV absorber.

The one or more haptics 104 can comprise or be made in part of a haptic material. The haptic material can comprise or be made in part of a cross-linked copolymer comprising a copolymer blend. The copolymer blend can comprise an alkyl acrylate, a fluoro-alkyl acrylate, and a phenyl-alkyl acrylate. For example, the cross-linked copolymer used to make the haptic material can comprise an alkyl acrylate in the amount of about 10% to 25% (wt %), a fluoro-alkyl acrylate in the amount of about 10% to 35% (wt %), and a phenyl-alkyl acrylate in the amount of about 50% to 80% (wt %). In some embodiments, the cross-linked copolymer used to make the haptic material can comprise n-butyl acrylate in the amount of about 10% to 25% (wt %) (e.g., between about 19% to about 23%), trifluoroethyl methacrylate in the amount of about 10% to 35% (wt %) (e.g., between about 14% to about 18%), and phenylethyl acrylate in the amount of about 50% to 80% (wt %) (e.g., between about 58% to about 62%). The final composition of the cross-linked copolymer used to make the haptic material can also comprise a cross-linker or cross-linking agent, such as EGDMA, in the amount of about 1.0%. The final composition of the cross-linked copolymer used to make the haptic material can also comprise a number of photoinitiators or photoinitiating agents (e.g., camphorquinone, 1-phenyl-1,2-propanedione, and 2-ethylhexyl-4-(dimenthylamino)benzoate).

In some embodiments, the refractive index of the lens body material can be between about 1.48 and about 1.53. In certain embodiments, the refractive index of the lens body material can be between about 1.50 and about 1.53 (e.g., about 1.5178).

The anterior element 130 can be attached or otherwise adhered to the posterior element 132 via adhesives 150 or an adhesive layer. The adhesive layer can be substantially annular-shaped. The adhesives 150 or adhesive layer can be positioned at a peripheral edge of the optic portion 102 in between the anterior element 130 and the posterior element 132. For example, the adhesives 150 can be positioned on top of the raised periphery 144 of the posterior element 132.

The adhesives 150 or adhesive layer can comprise or be made in part of a biocompatible adhesive. The adhesives 150 or adhesive layer can comprise or be made in part of a biocompatible polymeric adhesive.

The adhesives 150 or adhesive layer can comprise or be made in part of a cross-linkable polymer precursor formulation. The cross-linkable polymer precursor formulation can comprise or be made in part of a copolymer blend, a hydroxyl-functional acrylic monomer, and a photoinitiator.

The copolymer blend can comprise an alkyl acrylate (e.g., n-butyl acrylate in the amount of about 41% to about 45% (wt %)), a fluoro-alkyl acrylate (e.g., trifluoroethyl methacrylate in the amount of about 20% to about 24% (wt %)), and a phenyl-alkyl acrylate (phenylethyl acrylate in the amount of about 28% to about 32% (wt %)). The hydroxyl-functional acrylic monomer can be 2-hydroxyethyl acrylate (HEA). The photoinitiator can be used to facilitate curing of the adhesive. For example, the photoinitiator can be Darocur 4265 (a 50/50 blend of diphenyl(2,4,6-trimethylbenzoyl)phosphine oxide and 2-hydroxy2-methylpropiophenone).

In some embodiments, the same adhesives 150 used to bond the anterior element 130 to the posterior element 132 can also be used to bond or affix the one or more haptics 104 to the optic portion 102.

FIG. 1D also illustrates that each of the haptics 104 (e.g., any of the first haptic 104A or the second haptic 104B) can have a proximal attachment end 114 and a closed distal free end 116. A haptic fluid port 152 can be defined at the proximal attachment end 114 of the haptic 104. The haptic fluid port 152 can serve as a chamber opening of the haptic fluid lumen 106. Fluid within the haptic fluid lumen 106 can flow out of the haptic fluid lumen 106 through the haptic fluid port 152 and into the optic fluid chamber 108 via the pair of fluid channels 110 when the haptic 104 is coupled to the optic portion 102. Similarly, fluid within the optic fluid chamber 108 can flow out of the optic fluid chamber 108 through the pair of fluid channels 110 and into the haptic fluid lumen 106 through the haptic fluid port 152. A pair of outer apertures 156 and inner aperture 146 can serve as ends of the fluid channels 110.

As shown in FIGS. 1A and 1D, each of the haptics 104 can couple to the optic portion 102 at a reinforced portion 112. For example, the first haptic 104A can couple or be attached to the optic portion 102 at a first reinforced portion 112A and the second haptic 104B can couple or be attached to the optic portion 102 at the second reinforced portion 112B.

More specifically, the proximal attachment end 114 can couple to the protruding outer surface 154 of the posterior element 132. The protruding outer surface 154 can also be referred to as a “landing” or “haptic attachment landing.” The protruding outer surface 154 can extend out radially from an outer peripheral surface 122 of the optic portion 102. For example, the protruding outer surface 154 can extend out radially from an outer peripheral surface 122 of the posterior element 132 of the optic portion 102. The protruding outer surface 154 can extend out radially from the outer peripheral surface 122 between about 10 microns and 1.0 mm or between about 10 microns and 500 microns.

The proximal attachment end 114 can have a substantially flat surface to adhere or otherwise couple to a substantially flat surface of the protruding outer surface 154. When the proximal attachment end 114 is coupled to the protruding outer surface 154, the haptic fluid port 152 can surround the outer apertures 156 of the fluid channels 110. The haptics 104 can be coupled or adhered to the optic portion 102 via biocompatible adhesives 150. In some embodiments, the adhesives 150 can be the same adhesives used to couple or adhere the anterior element 130 to the posterior element 132.

FIG. 2A is a graphic representation of a composite material 200 comprising a composite base material 202, an energy absorbing constituent 204, and a plurality of expandable components 206. As previously discussed, one or more portions of each of the haptics 104 can be made of the composite material 200.

The composite base material 202 can be comprised of hydrophobic acrylic materials. For example, the composite base material 202 can be comprised of phenylethyl acrylate (PEA), a phenylethyl methacrylate (PEMA), or a combination thereof.

In one example embodiment, the composite base material 202 can comprise a methacrylate-functional or methacrylic-functional cross-linkable polymer and reactive acrylic monomer diluents including lauryl methacrylate (n-dodecyl methacrylate or SR313) and ADMA. By controlling the amount of lauryl methacrylate (SR313) to ADMA, the overall corresponding hardness (i.e., more ADMA) or softness (i.e., more SR313) of the cured composite material 200 can be controlled. The methacrylate-functional or methacrylic-functional cross-linkable polymer can be made using the cross-linkable polymer precursor formulation.

The cross-linkable polymer precursor formulation can comprise the same copolymer blend used to make the optic portion and the haptics.

The copolymer blend can comprise an alkyl acrylate or methacrylate (e.g., n-butyl acrylate), a fluoro-alkyl (meth)acrylate (e.g., trifluoroethyl methacrylate), and a phenyl-alkyl acrylate (e.g., phenylethyl acrylate). For example, the copolymer blend can comprise n-butyl acrylate in the amount of about 41% to about 45% (wt %), trifluoroethyl methacrylate in the amount of about 20% to about 24% (wt %), and phenylethyl acrylate in the amount of about 28% to about 32% (wt %). The cross-linkable polymer precursor formulation can comprise or be made in part of the copolymer blend, a hydroxyl-functional acrylic monomer (e.g., HEA), and a photoinitiator (e.g., Darocur 4265 or a 50/50 blend of diphenyl(2,4,6-trimethylbenzoyl)-phosphine oxide and 2-hydroxy2-methylpropiophenone).

The composite base material 202 can comprise the methacrylate-functional or methacrylic-functional cross-linkable polymer (as discussed above) in the amount of about 50% to about 65% (e.g., about 55% to about 60%) (wt %), the reactive acrylic monomer diluent lauryl methacrylate (SR313) in the amount of about 32% to about 38% (e.g., about 32.70%) (wt %), the reactive acrylic monomer diluent adamantly methacrylate (ADMA) in the amount of about 5% to about 9% (e.g., about 7.30%) (wt %).

Table 1 below provides an example formulation for the composite material 200:

TABLE 1
FORMULATION OF COMPOSITE MATERIAL (WT %)

| | |
|---|---|
| Cross-linkable polymer (in | 1.47% 2-hydroxyethyl acrylate (HEA) |
| two steps from precursor | 1.96% Darocur 4265 (photoinitiator) |
| formulation, as described | 43.49% n-butylacrylate (nBA) |
| above) | 30.21% 2-phenylethylacrylate (PEA) |
| | 22.87% 2,2,2-trifluoroethylmethacrylate |
| | (TFEMA) |
| Composite base material | 60.00% cross-linkable polymer |
| | 32.70% lauryl methacrylate (SR313) |
| | 7.30% 1-adamantyl methacrylate (ADMA) |
| Composite base material | 99.50% composite base material |
| with red energy absorbing | 0.50% Disperse Red 1 dye |
| colorant | |
| Composite base material | 99.95% composite base material |
| with black energy | 0.05% graphitized mesoporous carbon black |
| absorbing colorant | |
| Final formulation of | 87.70% composite base material with red or |
| composite material | black energy absorbing colorant |
| | 10.00% expandable microspheres |
| | 1.00% Luperox peroxide (thermal initiator) |
| | 1.30% Omnirad 2022 |

The composite material 200 can be made in several operations. The first operation can comprise preparing an uncolored composite base material 202. The second operation can comprise mixing the composite base material 202 with an energy absorbing constituent 204, expandable components 206, and initiators such as one or more photoinitiators, thermal initiators, or a combination thereof. The third operation can comprise placing the uncured composite material 200 into a desired location within the haptics 104 (e.g., in proximity to the channel 148), and curing the composite material 200 in place.

For example, the uncolored composite base material 202 can be mixed with an energy absorbing constituent 204 such as a dye (e.g., Disperse Red 1 dye) or pigment (graphitized carbon black). The energy absorbing constituent 204 will be discussed in more detail below.

In some embodiments, the expandable components 206 can make up about 5.0% to about 15.0% by weight of a final formulation of the composite material 200. More specifically, the expandable components 206 can make up about 8.0% to about 12.0% (e.g., about 10.0%) by weight of a final formulation (see Table 1) of the composite material 200. In these and other embodiments, the energy absorbing constituent 204 can make up about 0.044% to about 0.44% (or about 0.55%) by weight of the final formulation of the composite material 200.

The photoinitiator can be Omnirad 2022 (bis(2,4,6-trimethylbenzoyl)phenyl-phosphineoxide/2-hydroxy-2-methyl-1-phenyl-propan-1-one). The photoinitiator can make up about 1.30% by weight of a final formulation of the composite material 200 (see, e.g., Table 1). In addition, the composite material 200 can also comprise a thermal initiator. The thermal initiator can make up about 1.00% by weight of a final formulation of the composite material 200 (see, e.g., Table 1). In some embodiments, the thermal initiator can be a dialkyl peroxide such as Luperox® peroxide. In other embodiments, the thermal initiator can be Perkadox.

In some embodiments, the energy absorbing constituent 204 can absorb the external energy (e.g., laser energy), convert the energy to heat, and conduct the energy to the composite base material 202 to expand the composite base material 202.

FIG. 2B illustrates that the expandable components 206 can be expandable microspheres comprising an expandable thermoplastic shell 208 and a blowing agent 210 contained within the expandable thermoplastic shell 208. The microspheres can be configured to expand such that a diameter 212 of at least one of the microspheres can increase about 2× the original diameter. In other embodiments, the microspheres can be configured to expand such that the diameter 212 of at least one of the microspheres can increase about 4× or four times the original diameter. In further embodiments, the microspheres can be configured to expand such that the diameter 212 of at least one of the microspheres can increase between about 2× and about 4× (or about 3.5×) the original diameter. For example, the microspheres can have a diameter 212 of about 12 μm at the outset. In response to an external energy applied or directed at the composite material 200 or in response to energy transferred or transmitted to the microspheres, the diameter 212 of the microspheres can increase to about 40 μm.

The volume of at least one of the microspheres can be configured to expand between about ten times (10×) to about 50 times (50×) in response to the external energy applied or directed at the composite material 200 or in response to energy transferred or transmitted to the microspheres.

In some embodiments, the blowing agent 210 can be an expandable fluid, such as an expandable gas. More specifically, the blowing agent 210 can be a branched-chain hydrocarbon. For example, the blowing agent 210 can be isopentane. In other embodiments, the blowing agent 210 can be or comprise cyclopentane, pentane, or a mixture of cyclopentane, pentane, and isopentane.

The expandable components 206 can comprise differing amounts of the blowing agent 210. For example, some expandable components 206 can comprise more or a greater amount of the blowing agent (e.g., more expandable gas) to allow such expandable components 206 to expand more, resulting in greater expansion of the composite material 200 comprising such expandable components 206.

FIG. 2B illustrates that each of the expandable components 206 can comprise a thermoplastic shell 208. FIG. 2B also illustrates that a thickness of the thermoplastic shell 208 can change as the expandable component 206 increases in size. More specifically, the thickness of the thermoplastic shell 208 can decrease as the expandable component 206 increases in size. For example, when the expandable components 206 are expandable microspheres, the thickness of the thermoplastic shell 208 (i.e., its thickness in a radial direction) can decrease as the diameter 212 of the expandable microsphere increases.

For example, as previously discussed, at least one of the expandable microspheres can have a diameter 212 of about 12 μm at the outset. In this embodiment, the thermoplastic shell of the expandable microsphere can have a shell thickness of about 2.0 μm. In response to an external energy applied or directed at the composite material 200 or in response to energy transferred or transmitted to the microsphere, the diameter 212 of the microsphere can increase to about 40 μm (and the volume expand between about 10× and 50×) and the shell thickness of the microsphere can decrease to about 0.1 μm.

Although FIGS. 2A and 2B illustrate the expandable components 206 as spheres or microspheres, it is contemplated by this disclosure that the expandable components 206 can be substantially shaped as ovoids, ellipsoids, cuboids or other polyhedrons, or a combination thereof.

In some embodiments, the thermoplastic shell 208 can be made in part of nitriles or acrylonitrile copolymers. For example, the thermoplastic shell 208 can be made in part of acrylonitrile, styrene, butadiene, methyl acrylate, or a combination thereof.

As previously discussed, the expandable components 206 can make up between about 8.0% to about 12% by weight of a final formulation of the composite material 200. The expandable components 206 can make up about 10% by weight of a final formulation of the composite material 200.

The expandable components 206 can be dispersed or otherwise distributed within the composite base material 202 making up the bulk of the composite material 200. The composite base material 202 can serve as a matrix for holding or carrying the expandable components 206. The composite material 200 can expand in response to an expansion of the expandable components 206 (e.g., the thermoplastic microspheres). For example, a volume of the composite material 200 can increase in response to the expansion of the expandable components 206.

The composite material 200 also comprises an energy absorbing constituent 204. In some embodiments, the energy absorbing constituent 204 can be an energy absorbing colorant.

In certain embodiments, the energy absorbing colorant can be an energy absorbing dye. For example, the energy absorbing dye can be an azo dye. In some embodiments, the azo dye can be a red azo dye such as Disperse Red 1 dye. In other embodiments, the azo dye can be an orange azo dye such as Disperse Orange dye (e.g., Disperse Orange 1), a yellow azo dye such as Disperse Yellow dye (e.g., Disperse Yellow 1), a blue azo dye such as Disperse Blue dye (e.g., Disperse Blue 1), or a combination thereof.

In additional embodiments, the energy absorbing colorant can be or comprise a pigment. For example, the energy absorbing colorant can be or comprise graphitized carbon black as the pigment.

Similar to the expandable components 206, the energy absorbing constituent 204 can be dispersed or otherwise distributed within the composite base material 202 making up the bulk of the composite material 200. The composite base material 202 can serve as a matrix for holding or carrying the expandable components 206 and the energy absorbing constituent 204.

As previously discussed, the energy absorbing constituent 204 can make up between about 0.025% to about 1.0% (or, more specifically, about 0.045% to about 0.45%) by weight of a final formulation of the composite material 200. For example, when the energy absorbing constituent 204 is a dye (e.g., an azo dye such as Disperse Red 1), the energy absorbing constituent 204 can make up about between about 0.45% to about 1.0% by weight of a final formulation of the composite material 200. When the energy absorbing constituent 204 is graphitized carbon black or other types of pigments, the energy absorbing constituent 204 can make up about 0.025% to about 0.045% by weight of a final formulation of the composite material 200.

The energy absorbing constituent 204 (e.g., azo dye, graphitized carbon black, or a combination thereof) can absorb or capture an external energy (e.g., light energy or, more specifically, laser light) applied or directed at the composite material 200. The energy absorbing constituent 204 can absorb or capture the external energy and then transform or transfer the energy into thermal energy or heat to the expandable components 206.

The thermoplastic shell 208 can soften and begin to flow as thermal energy is transferred or transmitted to the expandable components 206. The thermoplastic shell 208 of the expandable components 206 can then begin to thin or reduce in thickness in response to the thermal energy transferred or transmitted to the expandable components 206. As the thermoplastic shell 208 begins to soften and reduce in thickness, the blowing agent 210 within the expandable components 206 can expand. The blowing agent 210 can also expand in response to the thermal energy or heat transferred or transmitted to the expandable components 206. Expansion of the blowing agents 210 can cause the expandable components 206 (e.g., the thermoplastic microspheres) to expand or increase in volume. This ultimately causes the composite material 200 to expand or increase in volume.

The composite material 200 can expand or increase in size in an isotropic manner such that the composite material 200 expands in all directions. Such isotropic expansion can be harnessed to produce expansion or material displacement in specific directions by placing or positioning the composite material 200 at specific locations within the haptic(s) 104 of the IOL 100.

As previously discussed, the external energy can be laser light 125 and the energy absorbing constituent 204 can absorb or capture the laser light 125 directed at the composite material 200 and transform or transfer the light energy into thermal energy or heat to the expandable components 206. The blowing agent 210 within the expandable components 206 can expand or become energized in response to the thermal energy or heat. The expandable components 206 and, ultimately, the composite material 200 can expand or increase in volume in response to this light energy directed at the composite material 200.

The shape change (e.g., increase in volume) undertaken by the expandable components 206 can be a persistent or a substantially permanent change. A persistent or substantially permanent change can mean that the expandable components 206 do not substantially revert back to its original shape or size after the shape change (e.g., after an increase in volume) has occurred. As a result, any change in the size or volume of the composite material 200 caused by a change in the size or volume of the expandable components 206 is also persistent or substantially permanent. As will be discussed in more detail in the following sections, this means that any structural changes made to the IOL 100 as a result of external energy or stimulus applied or otherwise directed at the composite material 200 embedded or integrated within the IOL 100 can persist or remain substantially permanent.

The thermoplastic shells 208 of the expandable components 206 can harden, once again, when the external energy is no longer directed or applied to the composite material 200. For example, the thermoplastic shells 208 may again harden when the temperature within a vicinity of the expandable components 206 falls below a certain threshold. For example, the thermoplastic shells 208 of the expandable microspheres can harden when light energy is no longer directed at the composite material 200. After the thermoplastic shells 208 harden, the expandable components 206 are locked into their new size and expanded configuration.

When the energy absorbing constituent 204 is an energy absorbing colorant, such as a dye or graphitized carbon, the color of at least part of the composite material 200 can take on the color of the energy absorbing colorant. For example, when the energy absorbing constituent 204 is an azo dye such as Disperse Red 1 having a red color, at least a portion of the composite material 200 comprising the energy absorbing constituent 204 can be colored red. Moreover, when the energy absorbing constituent 204 is graphitized carbon having a black color, at least a portion of the composite material 200 comprising the energy absorbing constituent 204 can be colored black. Although two colors (e.g., red and black) are mentioned in this disclosure, it is contemplated by this disclosure and it should be understood by one of ordinary skill in the art that energy absorbing colorant of other types of colors can also be used such as energy absorbing yellow, orange, or blue dyes or materials.

The color of the energy absorbing colorant can be visually perceptible to a clinician or another medical professional when at least part of the IOL 100 is made of the composite material 200 comprising the energy absorbing colorant. The color of the energy absorbing colorant can be visually perceptible to a clinician or another medical professional when the IOL 100 is implanted within an eye of a patient. For example, the composite material 200 can comprise Disperse Red 1 serving as the energy absorbing colorant. In this example, at least part of the IOL 100 can appear red to the clinician or another medical professional when the IOL 100 is implanted within the eye of a patient. The color of the energy absorbing colorant can allow the clinician or another medical professional to detect or determine the location or position of the composite material 200 within the IOL 100. The color of the energy absorbing colorant can also allow the clinician or another medical professional to determine where to direct the laser light 125 or stimulus to adjust the IOL 100.

However, in some instances, even when the composite material 200 is made of an energy absorbing colorant, the clinician or operator can have difficulty perceiving the color when the IOL 100 is implanted within the eye of the subject. Moreover, when different expandable components such as the lumen filler 126 and the lumen expander 128 are colored differently to set the structures apart, the clinician or operator may find it difficult to tell the colors apart. Therefore, a solution is needed to address these challenges and to ensure the safety of any post-implant adjustment procedures involving laser light.

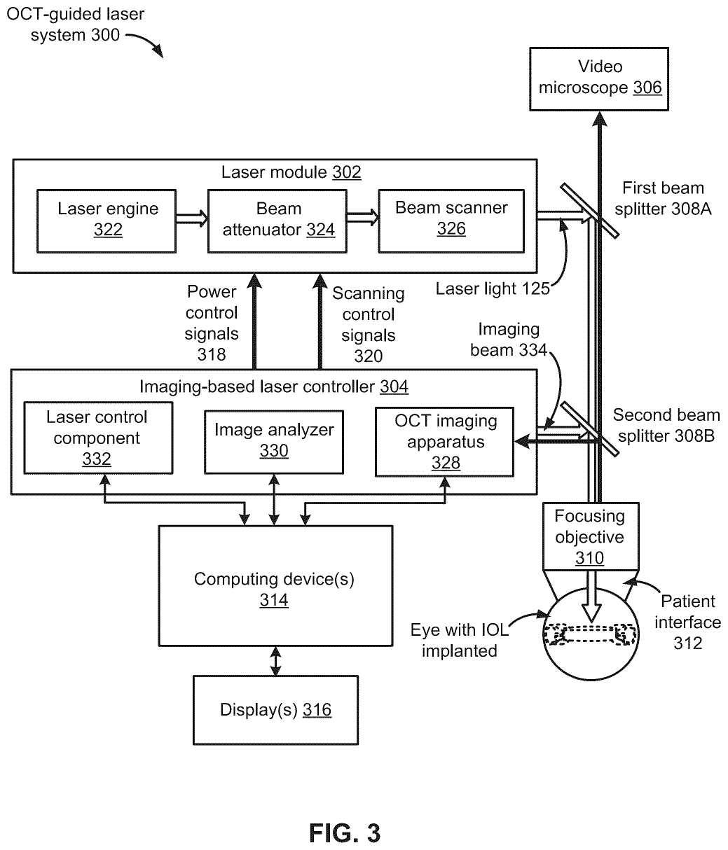

FIG. 3 is a block diagram illustrating one embodiment of an OCT-guided laser system 300. The system 300 can be used to guide a clinician during an IOL adjustment procedure where the aim of the procedure is to adjust a base power of an implanted IOL 100. For example, the system 300 can aid the clinician in precisely localizing IOL structures made of the composite material 200 and targeting such structures using laser light 125. More specifically, the system 300 can aid a clinician in precisely localizing the lumen fillers 126 and lumen expanders 128 and targeting such structures using laser light 125.

The system 300 can further aid the clinician in differentiating between different IOL structures made of the composite material 200 so as to avoid inadvertently targeting the wrong structure and causing an unintended power-change effect. For example, the system 300 can aid the clinician in differentiating between the lumen fillers 126 and the lumen expanders 128. This is important as the lumen fillers 126 and the lumen expanders 128 are often positioned close together or contiguous with one another.

In addition, the system 300 can allow the clinician to measure, in real-time, a net volume-change in IOL structures made of the composite material 200 once such structures have been exposed to laser light 125. These net volume-change measurements can then be used to calculate or estimate a resultant power change in the optic portion 102 of the IOL 100. Such real-time measurements can provide a closed-feedback loop where the clinician can adjust the amount of laser exposure, through controlling the number of laser pulses and/or the amount of energy per laser pulse, and/or the structure(s) targeted to achieve the desired power change.

The OCT-guided laser system 300 can also comprise specialized laser delivery optics (for example, a gonio lens 1000 integrated within a patient interface 312) that allow the laser light 125 to reach certain haptic structures made of the composite material 200 positioned radially outward of the optic portion 102.

As shown in FIG. 3, the OCT-guided laser system 300 can comprise a laser module 302, an imaging-based laser controller 304, a video microscope 306, a plurality of beam splitters including at least a first beam splitter 308A and a second beam splitter 308B, a focusing objective 310, a patient interface 312, one or more computing devices 314, and one or more displays 316.

The laser module 302 can generate and emit a beam of laser pulses to point(s) or focus spot(s) within the implanted IOL 100 as dictated by the imaging-based laser controller 304. The imaging-based laser controller 304 can image anatomical features of the eye along with devices implanted therein. The imaging-based laser controller 304 can also adjust certain beam parameters of the laser beam and control where the beam is directed within the eye. The imaging-based laser controller 304 can perform these functions by sending one or more power control signals 318 and scanning control signals 320 to the laser module 302.

The beam of laser light 125 or laser beam generated by the laser module 302 can be guided into the eye by a first beam splitter 308A. A focusing objective 310 can focus the laser beam using one or more objective lenses while the patient interface 312 can stabilize the eyeball of the patient. In some embodiments, the laser module 302 can generate the laser beam automatically based on beam parameters received via the power control signals 318 and the scanning control signals 320. In other embodiments, the laser module 302 can generate the laser beam based on beam parameters set, at least in part, by a clinician or operator via one of the computing devices 314.

The laser module 302 can comprise a laser engine 322, a beam attenuator 324, and a beam scanner 326. The laser engine 322 can be configured to generate the initial beam of laser pulses, the beam attenuator 324 can be configured to modify the beam of laser pulses based on the beam parameters, and the beam scanner 326 can be configured to direct the beam of laser pulses to one or more points or focus spots dictated by the imaging-based laser controller 304.

The laser engine 322 can comprise a solid-state laser source. More specifically, the laser engine 322 can comprise an oscillator configured to generate laser pulses (e.g., femtosecond laser pulses) with sufficient bandwidth, an amplifier configured to amplify the laser pulses to higher energies, and a compressor configured to compress the pulses back to a desired pulse range (e.g., to the femtosecond pulse range). In some embodiments, the amplifier can be a diode-pumped regenerative amplifier and the compressors can be a grating compressor. For example, the laser engine 322 can comprise an ultrafast pulsed diode-pumped solid-state femtosecond laser.

The laser engine 322 can generate laser pulses with a duration of nanoseconds (10⁻⁹ sec), picoseconds (10⁻¹² sec), or femtoseconds (10⁻⁵ sec). The laser pulses can be generated at a pulse repetition rate of between 1 kHz to up to 500 kHz. More specifically, the laser pulses can be generated at a pulse repetition rate of between about 10 kHz to about 100 kHz. In other embodiments, the laser pulses can be generated at a pulse repetition rate of between 0.1 kHz to 1,000 kHz.

In some embodiments, the laser beam generated by the laser engine 322 can have a wavelength of between about 900 nm and 1100 nm (i.e., in the near-infrared (NIR) range). For example, the laser beam generated by the laser engine 322 can have a wavelength of about 1030 nm.

In alternative embodiments, the laser beam generated by the laser engine 322 can be a green laser light with a wavelength between about 480 nm and 650 nm (e.g., 532 nm). In these embodiments, the laser engine 322 can comprise a neodymium-doped yttrium aluminum garnet (Nd:YAG) laser.

The beam attenuator 324 can be a computer-controlled attenuator that adjusts the beam energy to a desired level and continuously monitors the beam energy. The beam attenuator 324 can modify one or more beam parameters of the laser beam including a pulse energy, a pulse power, a pulse length, a pulse repetition rate, or a combination thereof. The beam attenuator can also comprise a shutter (e.g., a mechanical/electromechanical shutter) that can shutter or block selected laser pulses and/or a polarizer assembly that can reduce the power of selected laser pulses.

The beam scanner 326 can be configured to direct or focus the beam of laser pulses onto one or more focus spots at specific locations within the eye. For example, the beam scanner 326 can comprise a plurality of computer-controlled scanning mirrors that scan the laser beam into a beam-expander. Each scanned position of the laser beam corresponds to an X,Y location in a focal plane of the focusing objective 310. The Z-position of the focused laser spot can be computer controlled by optical zoom lenses located in the beam expander.

The imaging-based laser controller 304 can comprise an imaging system such as an OCT imaging apparatus 328, an image analyzer 330, and a laser control component 332. The OCT imaging apparatus 328 can generate an imaging beam 334 that is used to scan the eye of the subject including any implanted devices within the eye. The imaging beam 334 can be directed into the eye by the second beam splitter 308B. Light scattered from ocular anatomical structures and implant structures (e.g., haptics 104 and the optic portion 102 of the IOL 100 including any structures or elements thereof) can be reflected back to the imaging-based laser controller 304 via the second beam splitter 308B and used by the OCT imaging apparatus 328 to form cross-sectional images of the eye and any implants within the eye. The OCT imaging apparatus 328 can generate the cross-sectional images by measuring the echo time delay and intensity of the back-scattered or back-reflected light. The OCT measurements of echo time delays are based on correlation techniques that compare the back-scattered or back-reflected light signal against reference light signals traveling a known path length.

The OCT imaging apparatus 328 can comprise an OCT light source that generates light in a low-power visible wavelength. In other embodiments, the OCT light source can generate light in the near-infrared (NIR) range (i.e., at a wavelength between about 900 nm to about 1,400 nm).

For example, the OCT light source of the OCT imaging apparatus 328 can be a superluminescent diode. The OCT imaging apparatus 328 can also comprise an interferometer such as a Michelson interferometer, a reference mirror, galvoscanners used to scan the imaging beam 334 across the eye, and a spectrometer.

In some embodiments, the OCT imaging apparatus 328 can be communicatively coupled to one or more computing devices 314 that can be used to process imaging signals or data and control the various hardware components of the OCT imaging apparatus 328. OCT images generated by the OCT imaging apparatus 328 can also be presented to a clinician or an operator of the system 300 via one or more displays 316.

The OCT imaging apparatus 328 can be a spectral-domain OCT (SD-OCT). In other embodiments, the OCT imaging apparatus 328 can be a swept-source OCT (SS-OCT), a frequency domain OCT, a Fourier domain OCT, or a complex Fourier OCT.

The OCT imaging apparatus 328 can produce three-dimensional (3D) composite OCT images by combining two-dimensional (2D) cross-sectional OCT images. The 2D cross-sectional OCT images, the 3D OCT images, data associated with the imaging, or a combination thereof can be provided as inputs to the image analyzer 330. The image analyzer can be configured to analyze the 2D cross-sectional OCT images, the 3D composite OCT images, the data associated with the imaging, or a combination thereof to determine where certain structures or features made of the composite material 200 are located within the implanted IOL 100.

When the OCT imaging apparatus 328 is a spectral-domain OCT (SD-OCT), the OCT imaging apparatus 328 can produce an “en face” scan (a so-called “C-scan”) of the eye and any implants within the eye. An en face scan can be an OCT image of the eye and any implants within the eye taken along the coronal or front plane of the eye.

In some embodiments, the laser control component 332 can generate a set of X, Y, and Z-coordinates representing where the beam(s) of laser pulses generated by the laser module should be directed. The laser control component 332 can also set one or more beam parameters (e.g., a pulse energy, a pulse power, a pulse length, a pulse repetition rate, or a combination thereof) for each of the laser beams generated. The laser control component 332 can transmit the coordinates and the beam parameters to the laser module 302 via the power control signals 318 and the scanning control signals 320.

As will be discussed in more detail in later sections, the OCT imaging apparatus 328 can image the IOL structures targeted by the laser module 302 as well as other structures of the IOL after the IOL structures made of the composite material 200 (i.e., the targeted structures) are exposed to the beam(s) of laser light 125. For example, the OCT imaging apparatus 328 can produce any combination of 2D cross-sectional OCT images or 3D composite OCT images of the IOL structures after the structures made of the composite material 200 have been exposed to the beam(s) of the laser light 125. These 2D cross-sectional OCT images, 3D composite OCT images, and any data associated with the imaging can be provided as inputs to the image analyzer 330 to calculate a volume change of the IOL structures made of the composite material 200. As will be discussed in more detail in later sections, the image analyzer 330 can also be used to measure a change in at least one of a curvature of the anterior element 130, a curvature of the posterior element 132, and an axial thickness of the optic portion 102 of the IOL 100.

It is contemplated by this disclosure and it should be understood by one of ordinary skill in the art that any of the measurements or calculations undertaken by the image analyzer can also be undertaken by one or more computing devices 314 communicatively coupled to the imaging-based laser controller 304. In certain embodiments, the 2D cross-sectional OCT images, the 3D composite OCT images, or a combination thereof of any of the aforementioned IOL structures can be presented to a clinician or operator of the system 300 via one or more displays 316 (e.g., touchscreen displays or other type of computer displays). In these embodiments, the clinician or operator can perform certain calculations by measuring the IOL structures using the OCT images presented on the display(s) 316.

The computing device 314 can also be configured to determine a change in a base power of the IOL 100 based on the volume change of the composite material 200 in response to the beam of laser pulses directed at the composite material 200. For example, one or more processors of at least one of the computing devices 314 can be programmed to execute instructions stored on a memory unit or storage unit of the computing device 314 to determine the change in the base power of the IOL 100 based on the volume change of the composite material 200.

As previously discussed, one or more IOL structures (e.g., the lumen filler 126 and the lumen expander 128) can be made of the composite material 200 and the beam of laser pulses can be directed at these IOL structures. The volume change of the composite material 200 can be calculated by the image analyzer 330, the computing device(s) 314, or a combination thereof. As will be discussed in more detail in the following sections, in some embodiments, the computing device 314 can determine the change in the base power of the IOL 100 by first estimating a volume of fluid (e.g., silicone oil) displaced from either the haptic fluid lumen 106 to the optic fluid chamber 108 or the optic fluid chamber 108 to the haptic fluid lumen 106 in response to the volume change of the composite material 200 measured. The computing device 314 can then determine the change in the base power of the IOL 100 by selecting a base power change value from a readout table associated with the volume of fluid displaced.

The computing device 314 can also be configured to determine a change in a base power of the IOL 100 based on the measured change in at least one of the curvature of the anterior element 130, the curvature of the posterior element 132, and the axial thickness of the optic portion 102 of the IOL 100. As previously discussed, the image analyzer 330 can be configured to measure a change in at least one of the curvature of the anterior element 130, the curvature of the posterior element 132, and the axial thickness of the optic portion 102 of the IOL 100 based on OCT images (e.g., 2D cross-sectional OCT images and/or 3D composite OCT images) of the anterior element 130, the posterior element 132, and the optic portion 102 before and after an IOL structure made of the composite material 200 is exposed to the beam of laser light 125. As will be discussed in more detail in the following sections, in some embodiments, the computing device 314 can determine the change in the base power of the IOL 100 by selecting a base power change value from a readout table associated with the measured change in at least one of the curvature of the anterior element 130, the curvature of the posterior element 132, and the axial thickness of the optic portion 102.

In certain embodiments, the readout tables can be stored in a memory or storage unit of the computing device 314, the image analyzer 330, or a combination thereof. The readout tables can be constructed based on numerous laser-induced adjustment experiments conducted on the IOL 100 using different beam parameters. In these and other embodiments, multiple sets of readout tables can be stored on the computing device 314 and/or the image analyzer 330 with each set of readout tables associated with an IOL 100 having a unique combination of IOL structures made of the composite material 200 or IOL structures of different shapes or sizes.

In some embodiments, at least one of the image analyzer 330 and the computing device 314 can be configured to automatically differentiate between different IOL structures made of the composite material 200. For example, at least one of the image analyzer 330 and the computing device 314 can be configured to differentiate between the lumen filler 126 and the lumen expander 128 by analyzing the OCT images received as inputs from the OCT imaging apparatus 328. In other embodiments, at least one of the image analyzer 330 and the computing device 314 can provide suggestions or recommendations to a clinician or operator of the system 300 concerning whether an IOL structure is a lumen filler 126 or a lumen expander 128 with the clinician or operator making the final decision. In further embodiments, a display 316 of the system 300 can present OCT images captured by the OCT imaging apparatus 328 of different IOL structures and the clinician or operator can determine whether the IOL structure is a lumen filler 126 or a lumen expander 128 based on the OCT images displayed.

As previously discussed, in some embodiments, the system 300 can be designed as a closed-feedback loop where the laser control component 332 is configured to generate and transmit power control signals/commands 318 and scanning control signals/commands 320 to the laser module 302 to adjust certain beam parameter(s) and/or a target location based on a change in the base power of the IOL 100 determined by the computing device 314. In this manner, the imaging-based laser controller 304 can automatically control the laser module 302 without input from the clinician or operator.

It should be understood by one of ordinary skill in the art that even though various engines, modules, or components of the laser module 302 and the imaging-based laser controller 304 are shown as being separated from one another, such engines, modules, or components can be integrated with one another. For example, in some embodiments, the beam attenuator 324 can be part of the laser engine 322. In other embodiments, the beam scanner can be integrated with the beam attenuator 324. Moreover, in some embodiments, the image analyzer 330 can be integrated with or be part of the OCT imaging apparatus 328. In other embodiments, the laser control component 332 can be integrated with or be part of the image analyzer 330.

As shown in FIG. 3, the OCT-guided laser system 300 can further comprise a video microscope 306 that can capture video images of the eye and the implanted IOL 100 during the IOL adjustment procedure. The OCT-guided laser system 300 can also comprise a focusing objective 310 having an objective lens having a high numerical aperture (NA). For example, in some embodiments, the numerical aperture of the objective lens can be between about 0.2 and about 0.6.

The OCT-guided laser system 300 can also comprise a patient interface 312 that is configured to interface with the eye of the subject and stabilize the eye during the adjustment procedure. In some embodiments, the patient interface 312 can refer to an assembly comprising a patient interface lens and certain suction components. For example, the patient interface lens can be lowered or pressed onto the eye of the subject until the cornea of the subject is applanated. Suction is then activated to stabilize the eye and prevent it from moving during the procedure. The patient interface 312 can be mounted on a distal end of the focusing objective 310 and can serve as a sterile barrier between the eye of the subject and the remainder of the laser system 300.

In some embodiments, the patient interface 312 can also comprise a gonio lens 1000. The gonio lens 1000 can be configured to redirect the laser beam at part(s) of the IOL 100 made of the composite material 200 that is obscured by an anatomical structure of the eye. For example, the anatomical structure of the eye can be an iris of the eye and the part of the IOL 100 obscured by the iris can be a haptic 104 of the IOL 100. More specifically, the gonio lens 1000 can redirect the laser beam at parts of the haptic 104 made of the composite material 200 (e.g., the lumen filler 126 or the lumen expander 128) that are obscured by the iris of the eye.

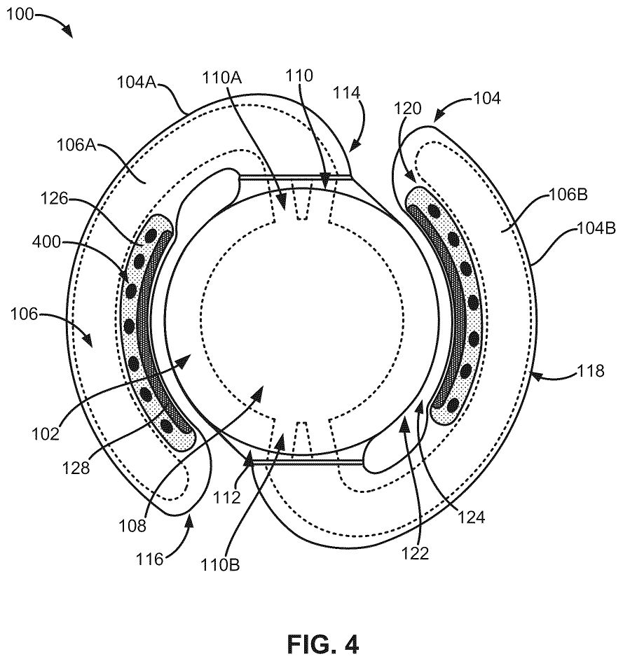

FIG. 4 illustrates a top-plan view (also referred to as an en face view) of the IOL 100 showing both lumen fillers 126 and lumen expanders 128 arranged along radially-inner haptic lumen walls 120 of the haptics 104. FIG. 4 also illustrates that beams of laser light 125 generated by the laser module 302 (see, e.g., FIG. 3) can be directed at multiple locations along the lumen fillers 126. Each location where the laser beam makes contact with the IOL 100 can be referred to as a laser spot 400.

As will be discussed in more detail in the following sections, a heated layer of the composite material 200 at each laser spot 400 can expand and cause a protuberance 500 (see, e.g., FIGS. 5B, 7A, and 7B) to form at the laser spot 400. The formation of protuberance(s) 500 can cause fluid within the haptic fluid lumen 106 to be displaced into the optic fluid chamber 108, thereby changing the base power of the optic portion 102.

In some embodiments, each laser spot 400 can be the result of one second of exposure to a beam of laser light 125. When the beam of laser light 125 has a pulse repetition rate of 50 kHz, the lumen filler 126 at the laser spot 400 can be exposed to 50,000 laser pulses with each such pulse having a pulse duration in the range of several hundred femtoseconds (e.g., 500 femtoseconds).

In some embodiments, the laser engine 322 can generate or adjust the pulse repetition rate of the laser light 125 to between about 1 kHz to up to 500 kHz. For example, the laser engine 322 can generate or adjust the pulse repetition rate of the laser light 125 to between about 10 kHz and about 100 kHz.

In certain embodiments, the laser engine 322 can generate or adjust the laser energy of the laser beam (i.e., the beam of laser light 125) to between about 0.1 μJ to about 100 μJ of laser energy per pulse. For example, the laser engine 322 can generate or adjust the laser energy of the laser beam to between about 1 μJ to about 50 μJ of laser energy per pulse.

As previously discussed, the laser module 302 can adjust the pulse repetition rate and/or the laser energy based on power control signals 318 received from the imaging-based laser controller 304 (see, e.g., FIG. 3). Moreover, the laser module 302 can adjust the pulse repetition rate and/or the laser energy based on inputs received from a clinician or operator via an operator interface such as a touchscreen display 316 or an input device communicatively coupled to the computing device 314.

Although FIG. 4 shows seven laser spots 400 along each of the lumen fillers 126, it is contemplated by this disclosure and it should be understood by one of ordinary skill in the art that each of the lumen fillers 126 (and lumen expanders 128) can accommodate more than seven laser spots. For example, each of the lumen fillers 126 and lumen expanders 128 can accommodate between 8 and 20 laser spots 400, or more than 20 laser spots 400.

In some embodiments, the location of each of the laser spots 400 can be calculated in advance by the computing device 314 based on OCT images of the haptic(s) 104 taken by the OCT imaging apparatus 328. For example, a target map or scan map can be created that indicates where each of the laser spots 400 should be located along the haptic(s) 104 of the IOL 100. In other embodiments, a clinician or operator can direct the laser module 302 in real-time to target locations selected by the clinician or operator based on real-time OCT images provided by the OCT imaging apparatus 328.

FIGS. 5A and 5B illustrate that a protuberance 500 can be formed at a laser spot 400 where the lumen filler 126 was exposed to the beam of laser light 125. As previously discussed, the lumen filler 126 can be made of the composite material 200. A heated layer 502 of the lumen filler 126 below the laser spot 400 can absorb the energy of the laser beam and expand in volume to form the protuberance 500. In certain embodiments, a depth of the heated layer 502 can be between about 0.05 mm to about 1.0 mm (e.g., about 0.10 mm).

Since the heated layer 502 is near the top or anterior surface of the lumen filler 126, the protuberance 500 can protrude or otherwise grow into the channel 148 of the haptic 104 (see, e.g., FIGS. 1B, 1C, and 7A). Since the channel 148 is also in fluid communication with the haptic fluid lumen 106, the formation of the protuberance 500 can displace fluid (e.g., silicone oil) from the haptic fluid lumen 106 into the optic fluid chamber 108. The amount of fluid displaced can be substantially or roughly equivalent to the volume of the protuberance 500.

One technical problem faced by the applicants is how to control the growth of the protuberance 500 using laser light 125. One technical solution discovered and developed by the applicants is to control the growth of the protuberance 500 by adjusting or varying at least one of: the laser energy per pulse, the pulse repetition rate, the size of the laser spot 400 or laser spot diameter 504, and a concentration of the energy absorbing pigment or dye.

For example, the size of the protuberance 500 can be fine-tuned by adjusting the size of the laser spot 400 or the laser spot diameter 504. The laser spot diameter 504 can be dependent on a cone angle 506 of the laser beam and an anteroposterior distance separating a focal point 508 of the laser beam from the laser spot 400 (which is also referred to as a focal point depth 510). As a more specific example, the laser spot diameter 504 can be dictated by Equation 1 below:

$$laser spot diameter = focal point depth * ( 2 * tan ( sin ( \frac{cone angle of laser beam}{2} ) ) ) \qquad [ Equation 1 ]$$

In some embodiments, the laser spot diameter 504 can be up to 1.0 mm. In other embodiments, the laser spot diameter 504 can be between about 0.5 mm and 1.0 mm. In additional embodiments, the laser spot diameter 504 can be between about 0.05 mm and 0.5 mm.

The protuberance 500 can also have a protuberance height 512. The protuberance height 512 can be the maxim height of the protuberance as measured from a top or anterior surface of the lumen filler 126 to an apex of the protuberance 500. In some embodiments, the protuberance height 512 can be up to 0.1 mm. In other embodiments, the protuberance height can be between about 0.05 mm and 0.1 mm. In additional embodiments, the protuberance height 512 can be between about 0.01 and 0.05 mm.

The volume of the protuberance 500 (and thereby, the volume of the fluid displaced and, ultimately, the change in the base power of the IOL 100) can be controlled by adjusting at least one of the laser energy per pulse, the pulse repetition rate, the cone angle 506 of the laser beam, and the focal point depth 510. Moreover, the composite material 200 itself can be made to be more responsive to the application of laser energy by increasing the concentration of the energy absorbing pigments or dyes within the composite material 200.

In some embodiments, the cone angle 506 of the laser beam can depend on the numerical aperture (NA) of the focusing objective 310 or objective lens used to focus the laser beam. For example, it has been discovered by the applicants that a focusing objective 310 or objective lens having a high numerical objective of between about 0.2 and 0.6 is more effective in causing a protuberance 500 to form and changing the base power of the IOL 100.

FIG. 6A to FIG. 6C illustrate various geometric shapes that the protuberance 500 can take when the protuberance 500 is fully formed. For example, FIG. 6A illustrates that the protuberance 500 can form into a hemisphere having a height dimension (hₕₑₘᵢₛₚₕₑᵣₑ). The hₕₑₘᵢₛₚₕₑᵣₑ can be the protuberance height 512. When the protuberance 500 is formed substantially as a hemisphere, the hₕₑₘᵢₛₚₕₑᵣₑ can be half of the laser spot diameter 504. Also, in this example, the volume of the hemispherical protuberance 500 can be calculated using Equation 2 below:

$$Volume of hemisphere protuberance ( {V}_{hemisphere} ) = ( 2 * π * {( {h}_{hemisphere} )}^{3} ) / 3 \qquad [ Equation 2 ]$$

FIG. 6B illustrates that the protuberance 500 can form into a paraboloid having a height dimension (h_{paraboloid}) and a width dimension (w_{paraboloid}). When the protuberance 500 is formed substantially as a paraboloid, the wₕₑₘᵢₛₚₕₑᵣₑ can be substantially equivalent to the laser spot diameter 504, and the h_{paraboloid} can be the protuberance height 512 or a maximum height or elevation of the paraboloid protuberance 500. In some embodiments, the maximum height/elevation of the paraboloid (as well as the wₕₑₘᵢₛₚₕₑᵣₑ) can be measured automatically by the image analyzer 330 or the computing device 314 (see, e.g., FIG. 3) based on 2D and 3D OCT images of the protuberance 500 provided by the OCT imaging apparatus 328. In other embodiments, a clinician or operator can measure the relevant dimensions of the protuberance 500 manually on screen using certain measuring tools or features provided as part of the OCT software. Also, in this example, the volume of the paraboloid protuberance 500 can be calculated using Equation 3 below:

Volume of paraboloid protuberance (V_{paraboloid})=(π*h_{paraboloid}*(w_{paraboloid})²)/8 [Equation 3]

FIG. 6C illustrates that the protuberance 500 can form into a half-ellipsoid having a height dimension (h_{ellipsoid}), a width dimension (w_{ellipsoid}), and a length dimension (l_{ellipsoid}). When the protuberance 500 is formed substantially as a half-ellipsoid, the w_{ellipsoid} and/or the l_{ellipsoid} can be substantially equivalent to the laser spot diameter 504 and the h_{ellipsoid} can be the protuberance height 512 or a maximum height or elevation of the half-ellipsoid protuberance 500. In some embodiments, the maximum height/elevation of the half-ellipsoid protuberance as well as any of the w_{ellipsoid} and the l_{ellipsoid} can be measured automatically by the image analyzer 330 or the computing device 314 (see, e.g., FIG. 3) based on 2D and 3D OCT images of the protuberance 500 provided by the OCT imaging apparatus 328. In other embodiments, a clinician or operator can measure the relevant dimensions of the protuberance 500 manually on screen using certain measuring tools or features provided as part of the OCT software. Also, in this example, the volume of the half-ellipsoid protuberance 500 can be calculated using Equation 4 below:

Volume of half-ellipsoid protuberance (V_{half-ellipsoid})=(π*h_{ellipsoid}*w_{ellipsoid}*l_{ellipsoid})/6 [Equation 4]

FIG. 7A is an OCT image showing a cross-section of a haptic 104 and part of the optic portion 102 of the IOL 100. As can be seen in FIG. 7A, a protuberance 500 is formed along a surface of the lumen filler 126 and protrudes or juts into the channel 148 of the haptic 104. Since the composite material 200 expands in a substantially isotropic manner when exposed to the laser beam, the heated composite material 200 expands in an anterior direction toward the channel 148 as well as in a posterior direction within the lumen filler 126. FIG. 7A highlights the contour or outer boundaries of this expansion.

As previously discussed, the channel 148 can be in fluid communication with the haptic fluid lumen 106 such that formation of the protuberance 500 can displace fluid (e.g., silicone oil) from the haptic fluid lumen 106 into the optic fluid chamber 108. It has been discovered by the applicants that the amount of fluid displaced can be substantially or roughly equivalent to the volume of the protuberance 500.

In some embodiments, the volume of the protuberance 500 can be calculated in real-time based on OCT images of the protuberance 500 such as the one shown in FIG. 7A. For example, a shape of the protuberance 500 can be approximated by comparing the shape against know geometric shapes such as a hemisphere, a paraboloid, or a half-ellipsoid. The relevant dimensions of the protuberance 500 can be measured based on the OCT image by the image analyzer 330/computing device 314 or measured on-screen by a clinician or operator.

The computing device 314 can then determine the change in the base power of the IOL by selecting a base power change value from a readout table associated with the volume of the fluid displaced. As previously mentioned, the computing device 314 can use the calculated volume of the protuberance 500 as an approximation for the volume of the fluid displaced. The readout tables can be stored in a memory or storage unit of the computing device 314, the image analyzer 330, or a combination thereof. The readout tables can be constructed based on laser-based adjustment experiments previously conducted on the IOL 100.

In some embodiments, one of the readout tables can associate a displacement of between about 10 nanoliter (nL) and 20 nL (e.g., about 15 nL) of fluid from the haptic fluid lumen 106 to the optic fluid chamber 108 with an approximately +0.10 diopter (D) change in the base power of the IOL 100. In this manner, applying laser light 125 to the lumen filler 126 can be considered plus activation as doing so increases the base power of the IOL 100.

FIG. 7B is an OCT image showing a cross-section of a segment of a lumen filler 126 with multiple protuberances 500 formed along the segment. For example, the protuberances 500 can be formed by targeting multiple spots along a length of the lumen filler 126. The multiple spots can be targeted in one post-implant adjustment procedure or in multiple post-implant adjustment procedures over time. The total volume of fluid displaced (from the haptic fluid lumen 106 to the optic fluid chamber 108) can be calculated based on the combined volume of the multiple protuberances 500.

FIG. 7C is an OCT image showing a cross-section of a haptic 104 and part of the optic portion 102 showing the lumen expander 128 in an expanded configuration. At least part of the lumen expander 128 composed of the composite material 200 can swell or expand in size to enlarge the lumen expander 128. When the lumen expander 128 is in the expanded configuration, a channel height 700 of the channel 148 can be increased along with a volume of the channel 148.

As previously discussed, and as can be seen in FIG. 7C, the lumen expander 128 can be positioned radially inward of the channel 148 and a radially outer lateral surface of the lumen expander 128 can be in fluid communication with the channel 148. The lumen expander 128 can be positioned in between the lumen filler 126 and an anterior portion of the radially-inner haptic lumen wall 120 in an anteroposterior direction. For example, an anterior end of the lumen expander 128 can adjoin and physically contact the anterior portion of the radially-inner haptic lumen wall 120 and a posterior end of the lumen expander 128 can adjoin and physically contact the lumen filler 126. Since the composite material 200 expands in a substantially isotropic manner when exposed to the laser beam, the lumen expander 128 in the expanded configuration pushes against the lumen filler 126 and the anterior portion of the radially-inner haptic lumen wall 120 to increase the channel height 700. Although part of the expansion can be in a radially inward direction (in a direction of the channel 148), any reduction in the volume of the channel 148 caused by the radially inward expansion is negligible and offset by the increase in the volume of the channel 148 caused by the increase in the channel height 700 in an anteroposterior direction.

As previously discussed, since the channel 148 is in fluid communication with the haptic fluid lumen 106 (or is considered part of the haptic fluid lumen 106), the volume of the haptic fluid lumen 106 can increase in response to the growth of the lumen expander 128. This can cause fluid within the haptic fluid lumen 106 to be drawn out of the optic fluid chamber 108 and into the haptic fluid lumen 106. The amount of fluid drawn back into the haptic fluid lumen 106 can be substantially or roughly equivalent to the increase in the volume of the channel 148.

The increase in the volume of the channel 148 can be calculated by measuring the channel height 700 before and after the lumen expander 128 is exposed to beam(s) of the laser light 125. These measurements can be done by analyzing OCT images taken of one or more cross-sections of the haptic 104 with the channel 148 visible in the OCT images. The OCT images can be taken before and after the lumen expander 128 is exposed to laser light 125. The increase in the volume of the channel 148 can then be calculated based on these OCT images. For example, the increase in the volume of the channel 148 can be calculated based on known static dimensions of the channel 148 and the change in the channel height 700. In other embodiments, the increase in the volume of the channel 148 can be calculated by measuring the dimensions of the channel 148 based on the OCT images or OCT imaging data. The dimensions of the channel 148 can be measured before and after the lumen expander 128 is exposed to the laser light 125.

The computing device 314 can then determine the change in the base power of the IOL 100 by selecting a base power change value from a readout table associated with the volume of the fluid drawn out of the optic fluid chamber 108 and into the haptic fluid lumen 106. As previously mentioned, the computing device 314 can use the calculated increase in the volume of the channel 148 as an approximation for the volume of the fluid drawn out of the optic fluid chamber 108 and into the haptic fluid lumen 106. The readout tables can be stored in a memory or storage unit of the computing device 314, the image analyzer 330, or a combination thereof. The readout tables can be constructed based on laser-based adjustment experiments previously conducted on the IOL 100.

In some embodiments, one of the readout tables can associate a fluid transfer or fluid displacement of between about 10 nanoliter (nL) and 20 nL (e.g., about 15 nL) from the optic fluid chamber 108 into the haptic fluid lumen 106 with an approximately −0.10 D change in the base power of the IOL 100. In this manner, applying laser light 125 to the lumen expander 128 can be considered negative activation as doing so decreases the base power of the IOL 100.

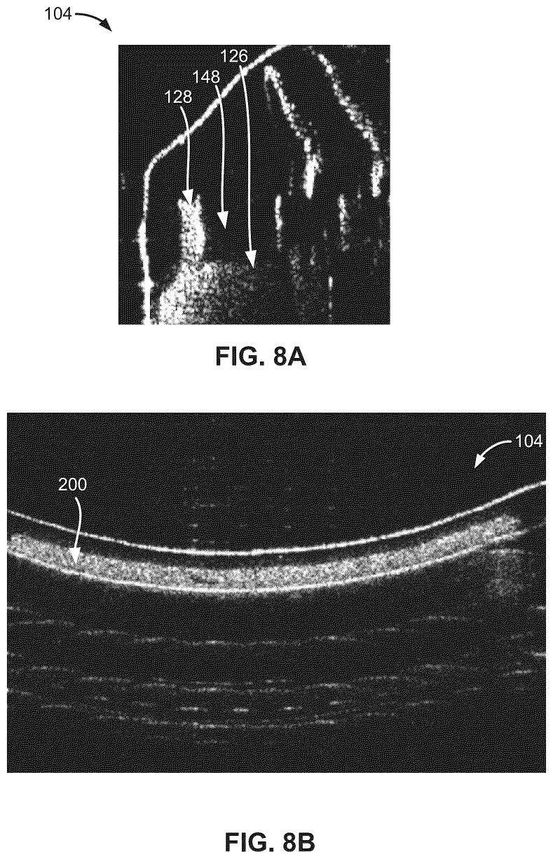

FIG. 8A is an OCT image showing a cross-section of a portion of a haptic 104 of the IOL 100 with a lumen filler 126 and a lumen expander 128 visible in the OCT image. FIG. 8B is an en face OCT image showing a top-down view of a portion of the haptic 104 of the IOL 100 with a segment of the haptic 104 made of the composite material 200 visible in the en face OCT image.

As previously discussed, a method of adjusting the implanted IOL 100 can comprise using the OCT imaging apparatus 328 to image the implanted IOL 100 prior to targeting the lumen filler 126 or the lumen expander 128 with beam(s) of laser light 125. In some embodiments, at least one of the computing device 314 and the image analyzer 330 can automatically determine a location of the lumen filler 126 or the lumen expander 128 based on one or more OCT images of the IOL 100 or OCT imaging data obtained from the OCT imaging apparatus 328. In other embodiments, a clinician or operator can determine the location of the lumen filler 126 or the lumen expander 128 by examining the OCT images of the IOL 100.

As shown in FIGS. 8A and 8B, the composite material 200 (in the form of either the lumen filler 126 or the lumen expander 128) is distinctly visible in both the cross-sectional and en face OCT images. This can be due to differences in the density of the composite material 200 compared to the surrounding haptic material and fluid within the haptic fluid lumen 106. As previously mentioned, the composite material 200 can be comprised of certain energy-absorbing pigments/dyes and the expandable microspheres, all of which can contribute to the visibility of the composite material 200 under OCT.

The computing device 314 can initially use enface OCT images to determine X- and Y-coordinates of the composite material 200 and then use the cross-sectional OCT images to determine Z-coordinates or depth coordinates of the composite material 200.

The computing device 314 can also use the cross-sectional OCT images to differentiate between the lumen filler 126 and the lumen expander 128. For example, the computing device can differentiate between the lumen filler 126 and the lumen expander 128 based on the location of such structures relative to other haptic structures (where the shapes and sizes of such other haptic structures remain static and are well documented) and based on their locations relative to one another. As a more specific example, the lumen filler 126 can be differentiated from the lumen expander 128 based on the presence of fluid within the channel immediately anterior to the lumen filler 126.

One technical problem faced by the applicant is how to precisely target only one of the lumen filler 126 or the lumen expander 128 with laser beam(s) without inadvertently hitting the other structure. This is especially difficult given that both the lumen filler 126 and the lumen expander 128 are arranged along curved regions of the haptic(s) 104 and a single segment of either the lumen filler 126 or the lumen expander 128 may be positioned at different depths due to the fact that an implanted IOL 100 is often tilted at an angle (called the angle kappa) with respect to the visual axis of the eye (which is also the case for the subject's own natural lens). One technical solution discovered and developed by the applicants is to image the haptic 104 containing the lumen filler 126 and the lumen expander 128 using OCT and determine the X, Y, and Z-coordinates of the lumen filler 126 and the lumen expander 128 based on the OCT images. A scan pattern or target map can be created that includes the X, Y, and Z-coordinates of the intended laser spots along either the lumen filler 126 or the lumen expander 128. The laser beams can then be applied in accordance with the scan pattern or the target map so as to prevent the laser beams from inadvertently targeting the wrong expandable structure or inadvertently harming the eye of the subject.

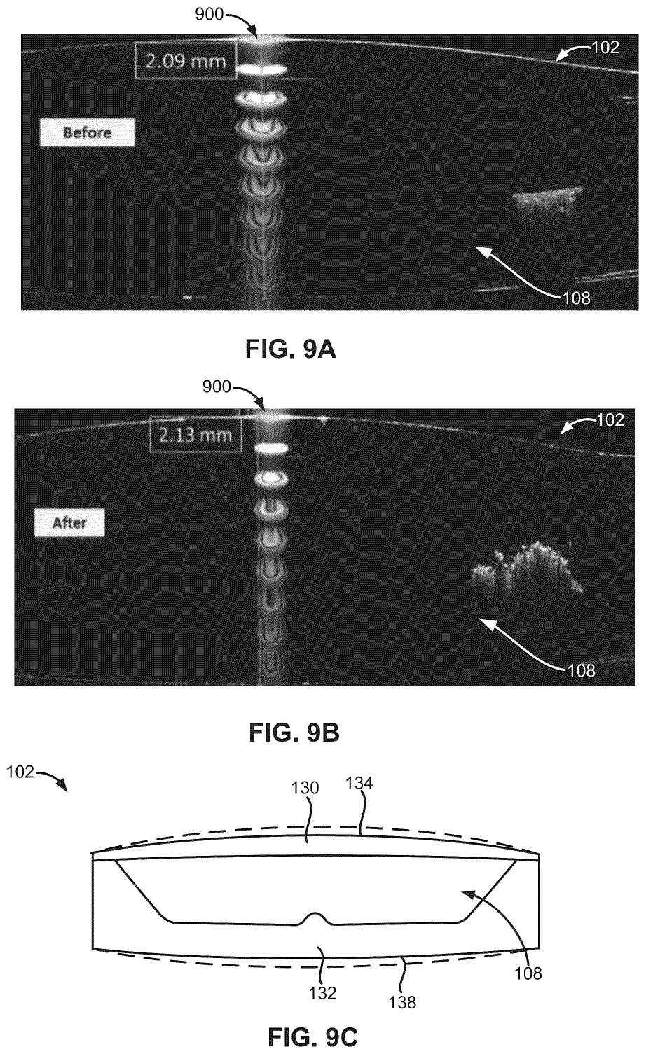

FIGS. 9A and 9B are before-and-after OCT images showing a change in an axial thickness 900 of the optic portion 102 in response to fluid entering the optic fluid chamber 108. As previously discussed, the optic portion 102 can undergo a shape change in response to fluid entering and exiting the optic fluid chamber 108. For example, as shown in FIGS. 9A and 9B, the axial thickness 900 of the optic portion 102 can increase by 0.04 mm in response to a beam of laser light 125 applied to the lumen filler 126. The laser light 125 can cause a protuberance 500 to form at a laser spot 400 along the lumen filler 126 and the protuberance 500 can cause fluid within the haptic fluid lumen 106 to be displaced into the optic fluid chamber 108.

FIG. 9C illustrates that at least one of the anterior element 130 and the posterior element 132 can change its curvature in response to fluid entering or exiting the optic fluid chamber 108. As a more specific example, at least one of the anterior optical surface 134 of the anterior element 130 and the posterior optical surface 138 of the posterior element 132 can be configured to increase its curvature in response to fluid entering the optic fluid chamber 108 and at least one of the anterior optical surface 134 of the anterior element 130 and the posterior optical surface 138 of the posterior element 132 can be configured to decrease its curvature (or flatten out) in response to fluid exiting the optic fluid chamber 108.

The axial thickness 900 can be a distance separating the anterior-most point along the anterior optical surface 134 and a posterior-most point along the posterior optical surface 138 as measured along the optical axis 142. When at least one of the anterior element 130 and the posterior element 132 increases its curvature, the axial thickness 900 of the optic portion 102 can increase. On the contrary, when at least one of the anterior element 130 and the posterior element 132 decreases its curvature, the axial thickness 900 of the optic portion 102 can decrease.

In some embodiments, at least one of the image analyzer 330 and the computing device can automatically measure a change in at least one of the axial thickness 900, the curvature of the anterior optical surface 134, and the curvature of the posterior optical surface 138 based on one or more cross-sectional OCT images of the IOL 100 or OCT imaging data obtained from the OCT imaging apparatus 328. In other embodiments, a clinician or operator can measure the change in at least one of the axial thickness 900, the curvature of the anterior optical surface 134, and the curvature of the posterior optical surface 138 by examining one or more cross-sectional OCT images.

In some embodiments, the computing device 314 can be configured to determine a change in a base power of the IOL 100 based on the measured change in at least one of the curvature of the anterior element 130, the curvature of the posterior element 132, and the axial thickness 900 of the optic portion 102. For example, the computing device 314 can be configured to determine the change in the base power of the IOL 100 by selecting a base power change value associated with the measured change in at least one of the curvature of the anterior element 130, the curvature of the posterior element 132, and the axial thickness 900 from one or more readout tables. Also, for example, the computing device 314 can be configured to determine the change in the base power of the IOL 100 using a mathematical relationship where the change in the axial thickness 900 is multiplied by a conversion factor. As a more specific example, the conversion factor can be an approximately 60 D (±5 D) change per millimeter change in the axial thickness 900.

FIG. 10 illustrates that a gonio lens 1000 can be used to re-direct laser light 125 to a part of the IOL 100 made up of the composite material 200 that is obscured by an anatomical structure of the eye. In some embodiments, the gonio lens 1000 can be incorporated or integrated into the patient interface 312 that physically contacts the eye of the subject. In certain embodiments, numbing drops are applied to the eye of the subject before the gonio lens 1000 makes contact with the eye.

The gonio lens 1000 can comprise a number of angled mirrors 1002 arranged within an interior of a lens housing of the gonio lens 1000. In some embodiments, the gonio lens 1000 can comprise between two and six angled mirrors 1002. In other embodiments, the gonio lens 1000 can comprise only one angled mirror 1002 or more than six angled mirrors 1002 such as between eight and ten angled mirrors 1002.

In some embodiments, each of the angled mirrors 1002 of the gonio lens 1000 can be positioned at a different angle within the lens housing. In other embodiments, at least two of the angled mirrors 1002 can be positioned at the same angle. For example, each of the angled mirrors 1002 can be positioned at an angle between 58° and 80°.

As shown in FIG. 10, the angled mirror 1002 of the gonio lens 1000 can redirect the laser beam to parts of the implanted IOL 100 that are obscured by an anatomical structure of the eye. For example, the angled mirror 1002 of the gonio lens 1000 can redirect the laser beam to parts of the implanted IOL 100 that are obscured by the iris of the eye. As a more specific example, the angled mirror 1002 of the gonio lens 1000 can redirect the laser beam to parts of the haptics 104 (e.g., parts of the lumen filler 126 or the lumen expander 128) of the implanted IOL 100 that are obscured by the iris of the eye.

The gonio lens 1000 can contribute to the improved safety of the system 300. For example, one technical problem faced by the applicant is the risk of inadvertently damaging the patient's eye via exposure to laser energy. This risk is enhanced when such laser energy must be directed at one or more haptics 104 located peripheral to a central optic portion 102. As a more specific example, the patient's iris can be inadvertently exposed to laser energy while the laser is being aimed at peripheral regions outside of the central optic portion 102. The gonio lens 1000 can contribute to the improved safety of the system 300 by allowing the system 300 to precisely target parts of the IOL 100 positioned peripheral to the central optic portion 102 without inadvertently harming the eye. For example, the gonio lens 1000 can prevent the iris from being inadvertently exposed to the laser beam.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of”, when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially”, “about” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1. A method of adjusting an intraocular lens (IOL) with optical coherence tomography (OCT) guidance, comprising:
directing a laser beam generated by a laser system at a composite material making up part of the IOL, wherein at least part of the composite material expands in volume in response to the laser beam directed at the composite material;
measuring a volume change of the composite material by analyzing one or more OCT images of the composite material produced by an OCT imaging apparatus; and
determining a change in a base power of the IOL based on the volume change of the composite material measured.

2. The method of claim 1, further comprising:
imaging the IOL comprising the composite material using the OCT imaging apparatus prior to directing the laser beam at the composite material; and
determining a location of the composite material based on the OCT imaging.

3. The method of claim 2, wherein the IOL comprises at least one haptic comprising a haptic fluid lumen and a radially-inner haptic lumen wall surrounding at least part of the haptic fluid lumen,
wherein the composite material is configured as a lumen filler making up part of the radially-inner haptic lumen wall and wherein the lumen filler is configured to expand into at least part of the haptic fluid lumen to reduce a volume of the haptic fluid lumen in response to the laser beam directed at the lumen filler, and
wherein the composite material is also configured as a lumen expander making up another part of the radially-inner haptic lumen wall and wherein the lumen expander is configured to expand to increase the volume of the haptic fluid lumen in response to the laser beam directed at the lumen expander; and
wherein the method further comprises differentiating between the lumen filler and the lumen expander by analyzing the one or more OCT images.

4. The method of claim 1, further comprising adjusting a pulse repetition rate of the laser beam to between about 10 kHz to about 100 kHz.

5. The method of claim 1, further comprising adjusting a laser energy of the laser beam to between about 0.1 μJ to about 100 μJ of laser energy per pulse.

6. The method of claim 1, further comprising controlling the volume change of the composite material by controlling a laser spot diameter created by the laser beam on the composite material, wherein the laser spot diameter is dictated by the relationship:
$$laser spot diameter = focal point depth * ( 2 * tan ( sin ( \frac{cone angle of laser beam}{2} ) ) ) .$$

7. The method of claim 1, wherein the laser beam has a wavelength of between about 1030 nm to about 1064 nm.

8. The method of claim 1, wherein the laser beam is focused by a focusing objective having a numerical aperture of between 0.2 and 0.6, and wherein the laser beam is focused by the focusing objective onto the composite material.

9. The method of claim 1, further comprising redirecting the laser beam at the composite material using a gonio lens such that the laser beam reaches a part of the IOL obscured by an anatomical structure of the eye.

10. The method of claim 1, wherein determining the change in the base power of the IOL further comprises estimating a volume of fluid displaced from either a haptic fluid lumen to an optic fluid chamber or the optic fluid chamber to the haptic fluid lumen in response to the volume change of the composite material measured, and determining the change in the base power by selecting a base power change value associated with the volume of fluid displaced from a readout table.

11. A method of adjusting an intraocular lens (IOL) with optical coherence tomography (OCT) guidance, comprising:
directing a laser beam generated by a laser system at a composite material making up part of the IOL,
wherein at least part of the composite material expands in volume in response to the laser beam directed at the composite material;
wherein the IOL comprises an optic portion comprising an anterior element and a posterior element;
measuring a change in at least one of a curvature of the anterior element, a curvature of the posterior element, and an axial thickness of the optic portion by analyzing one or more OCT images of the optic portion produced by an OCT imaging apparatus; and
determining a change in a base power of the IOL based on the measured change in at least one of the curvature of the anterior element, the curvature of the posterior element, and the axial thickness of the optic portion.

12. The method of claim 11, further comprising:
imaging the IOL comprising the composite material using the OCT imaging apparatus prior to directing the laser beam at the composite material; and
determining a location of the composite material based on the OCT imaging.

13. The method of claim 12, wherein the IOL comprises at least one haptic comprising a haptic fluid lumen and a radially-inner haptic lumen wall surrounding at least part of the haptic fluid lumen,
wherein the composite material is configured as a lumen filler making up part of the radially-inner haptic lumen wall and wherein the lumen filler is configured to expand into at least part of the haptic fluid lumen to reduce a volume of the haptic fluid lumen in response to the laser beam directed at the lumen filler, and
wherein the composite material is also configured as a lumen expander making up another part of the radially-inner haptic lumen wall and wherein the lumen expander is configured to expand to increase the volume of the haptic fluid lumen in response to the laser beam directed at the lumen expander; and
wherein the method further comprises differentiating between the lumen filler and the lumen expander by analyzing the one or more OCT images.

14. The method of claim 11, further comprising adjusting a pulse repetition rate of the laser beam to between about 10 kHz to about 100 kHz.

15. The method of claim 11, further comprising adjusting a laser energy of the laser beam to between about 0.1 μJ to about 100 μJ of laser energy per pulse.

16. The method of claim 11, further comprising controlling the volume change of the composite material by controlling a laser spot diameter created by the laser beam on the composite material, wherein the laser spot diameter is dictated by the relationship:
$$laser spot diameter = focal point depth * ( 2 * tan ( sin ( \frac{cone angle of laser beam}{2} ) ) ) .$$

17. (canceled)

18. (canceled)

19. (canceled)

20. (canceled)

21. An ophthalmic system, comprising:
a laser system configured to generate a laser beam directed at a composite material making up part of an intraocular lens (IOL), wherein at least part of the composite material expands in volume in response to the laser beam directed at the composite material;
an optical coherence tomography (OCT) imaging apparatus configured to produce one or more OCT images of the composite material making up part of the IOL;
an image analyzer configured to measure a volume change of the composite material by analyzing the one or more OCT images;
a computing device configured to determine a change in a base power of the IOL based on the volume change of the composite material.

22. (canceled)

23. (canceled)

24. (canceled)

25. (canceled)

26. (canceled)

27. (canceled)

28. (canceled)

29. (canceled)

30. (canceled)

31. An ophthalmic system, comprising:
a laser system configured to generate a laser beam directed at a composite material making up part of an intraocular lens (IOL),
wherein at least part of the composite material expands in volume in response to the laser beam directed at the composite material,
wherein the IOL comprises an optic portion comprising an anterior element and a posterior element;
an optical coherence tomography (OCT) imaging apparatus configured to produce one or more OCT images of the optic portion of the IOL after the laser beam is directed at the composite material;
an image analyzer configured to measure a change in at least one of a curvature of the anterior element, a curvature of the posterior element, and an axial thickness of the optic portion by analyzing the one or more OCT images of the optic portion; and
a computing device configured to determine a change in a base power of the IOL based on the measured change in at least one of the curvature of the anterior element, the curvature of the posterior element, and the axial thickness of the optic portion.

32. (canceled)

33. (canceled)

34. (canceled)

35. (canceled)

36. (canceled)

37. (canceled)

38. (canceled)

39. (canceled)

40. (canceled)

41. A method of adjusting an intraocular lens (IOL) with optical coherence tomography (OCT) guidance, comprising:
directing a laser beam generated by a laser system at a composite material making up part of the IOL, wherein at least part of the composite material expands in volume in response to the laser beam directed at the composite material;
measuring a volume change of a structure or cavity within the IOL in response to the expansion of the composite material by analyzing OCT images of the IOL produced by an OCT imaging apparatus; and
determining a change in a base power of the IOL based on the volume change of the structure or cavity measured.

42. (canceled)

43. (canceled)

44. (canceled)

45. (canceled)

46. (canceled)

47. (canceled)

48. (canceled)

49. (canceled)

50. (canceled)

51. An ophthalmic system, comprising:
a laser system configured to generate a laser beam directed at a composite material making up part of an intraocular lens (IOL), wherein at least part of the composite material expands in volume in response to the laser beam directed at the composite material;
an optical coherence tomography (OCT) imaging apparatus configured to produce OCT images of the IOL including a structure or cavity within the IOL;
an image analyzer configured to measure a volume change of the structure or cavity within the IOL in response to the expansion of the composite material by analyzing the OCT images;
a computing device configured to determine a change in a base power of the IOL based on a volume change of the structure or cavity measured.

52. (canceled)

53. (canceled)

54. (canceled)

55. (canceled)

56. (canceled)

57. (canceled)

58. (canceled)

59. (canceled)

60. (canceled)
